SEQUENCE LISTING (27 sequences)

SEQ ID NO: 1 - DNA, 447 bp, synthetic construct
  source 1..447: mol_type = other DNA
cagcactgaaggcgcgctgacgtcactcgccggtcccccgcaaactccccttcccggccaccttggtcgcgtccgcgccgccgccggcccagccggaccgcaccacgcgaggcgcgagataggggggcaccagcactgaaggcgcgctgacgtcactcgccggtcccccgcaaactccccttcccggccaccttggtcgcgtccgcgccgccgccggcccagccggaccgcaccacgcgaggcgcgagataggggggcaccagcactgaaggcgcgctgacgtcactcgccggtcccccgcaaactccccttcccggccaccttggtcgcgtccgcgccgccgccggcccagccggaccgcaccacgcgaggcgcgagataggggggcacagctcgggctgggcataaaagtcagggcagagccatctattgcttacatttgcttct

SEQ ID NO: 2 - DNA, 537 bp, synthetic construct
  source 1..537: mol_type = other DNA
cccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccagctcgggctgggcataaaagtcagggcagagccatctattgcttacatttgcttct

SEQ ID NO: 3 - DNA, 217 bp, synthetic construct
  source 1..217: mol_type = other DNA
cccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccagctcgggctgggcataaaagtcagggcagagccatctattgcttacatttgcttct

SEQ ID NO: 4 - DNA, 624 bp, synthetic construct
  source 1..624: mol_type = other DNA
ggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 5 - DNA, 531 bp, synthetic construct
  source 1..531: mol_type = other DNA
gggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggccccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggtgaggcccgtatcgggccgcatccccttactctgtccttgccgcgtccactccccgttatcctgggcttagcgcccccgggtgtgtgtgtgtgtgtgtgtgtgtgtgtgtgtgtgtgtgtgtgtgtgtgtgtgtgtgtgacgctgcgtacctgtca

SEQ ID NO: 6 - DNA, 452 bp, synthetic construct
  source 1..452: mol_type = other DNA
tcccgcaaccccccccccccccacatgtcctctgtcttctgctgccacaaagggttaatctctcctgcgggtaaaaacaggtccgcggagtctctaactggcgacagatgggccactttcttctggccacaaaggggccggaatggagcgctccgcggcatacaaataggcaggtcacgtggttcccggctcttggctggaccgggaagaccatatggcgcatgccggggaggaaggagcaggggcggaggtactgtgggggtgaggggagtggtggtggaagggggcgggaggaaagttctggggaggggtgaatgagggcgggagtcgttcagtctggacgcgtgcgcgattgcggggcgcagggacctgctcgttgctcagctcacggccccgcccgcgctcagcatcagcaactcggctatataaccctagcgcccgcgccaccggga

SEQ ID NO: 7 - DNA, 629 bp, synthetic construct
  source 1..629: mol_type = other DNA
cccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccagctcgggctgggcataaaagtcagggcagagccatctattgcttacatttgcttctaagaggtaagggtttaagggatggttggttggtggggtattaatgtttaattacctggagcacctgcctgaaatcactttttttcaggttgg

SEQ ID NO: 8 - DNA, 617 bp, synthetic construct
  source 1..617: mol_type = other DNA
acccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgcagctcgggctgggcataaaagtcagggcagagccatctattgcttacatttgcttct

SEQ ID NO: 9 - DNA, 587 bp, synthetic construct
  source 1..587: mol_type = other DNA
atcagagagggggaggggaaacaggatgcggcgaggcgcgtgcgcactgccagcttcagcaccgcggacagtgccttcgcccccgcctggcggcgcgcgccaccgcatcagagagggggaggggaaacaggatgcggcgaggcgcgtgcgcactgccagcttcagcaccgcggacagtgccttcgcccccgcctggcggcgcgcgccaccgcatcagagagggggaggggaaacaggatgcggcgaggcgcgtgcgcactgccagcttcagcaccgcggacagtgccttcgcccccgcctggcggcgcgcgccaccgcatcagagagggggaggggaaacaggatgcggcgaggcgcgtgcgcactgccagcttcagcaccgcggacagtgccttcgcccccgcctggcggcgcgcgccaccgcatcagagagggggaggggaaacaggatgcggcgaggcgcgtgcgcactgccagcttcagcaccgcggacagtgccttcgcccccgcctggcggcgcgcgccaccgcagctcgggctgggcataaaagtcagggcagagccatctattgcttacatttgcttct

SEQ ID NO: 10 - DNA, 537 bp, synthetic construct
  source 1..537: mol_type = other DNA
gaaggcgcgctgacgtcactcgccggtcccccgcaaactccccttcccggccaccttggtcgcgtccgcgccgccgccgggaaggcgcgctgacgtcactcgccggtcccccgcaaactccccttcccggccaccttggtcgcgtccgcgccgccgccgggaaggcgcgctgacgtcactcgccggtcccccgcaaactccccttcccggccaccttggtcgcgtccgcgccgccgccgggaaggcgcgctgacgtcactcgccggtcccccgcaaactccccttcccggccaccttggtcgcgtccgcgccgccgccgggaaggcgcgctgacgtcactcgccggtcccccgcaaactccccttcccggccaccttggtcgcgtccgcgccgccgccgggaaggcgcgctgacgtcactcgccggtcccccgcaaactccccttcccggccaccttggtcgcgtccgcgccgccgccggagctcgggctgggcataaaagtcagggcagagccatctattgcttacatttgcttct

SEQ ID NO: 11 - DNA, 523 bp, synthetic construct
  source 1..523: mol_type = other DNA
ggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagcc

SEQ ID NO: 12 - DNA, 627 bp, synthetic construct
  source 1..627: mol_type = other DNA
gaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatcgaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatcagctcgggctgggcataaaagtcagggcagagccatctattgcttacatttgcttct

SEQ ID NO: 13 - DNA, 525 bp, synthetic construct
  source 1..525: mol_type = other DNA
acacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagcc

SEQ ID NO: 14 - DNA, 88 bp, synthetic construct
  source 1..88: mol_type = other DNA
ggcggggcccggtcgcccctccccctccccgccctcctgcgccgggagcagtgcattgtgggaaactcccgagctctcctccgcgttc

SEQ ID NO: 15 - DNA, 252 bp, synthetic construct
  source 1..252: mol_type = other DNA
ggcccggtcgcccctccccctccccgccctcctgcgccgggagcagtgcattgtgggaaactcccggcccggtcgcccctccccctccccgccctcctgcgccgggagcagtgcattgtgggaaactcccggcccggtcgcccctccccctccccgccctcctgcgccgggagcagtgcattgtgggaaactcccagctcgggctgggcataaaagtcagggcagagccatctattgcttacatttgcttct

SEQ ID NO: 16 - DNA, 733 bp, synthetic construct
  source 1..733: mol_type = other DNA
tgacaatgctccctctggcctcactcccttttatacgtacttccatcaaaccatctgattcaacaatgacagaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 17 - DNA, 361 bp, synthetic construct
  source 1..361: mol_type = other DNA
agacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 18 - DNA, 82 bp, synthetic construct
  source 1..82: mol_type = other DNA
aacttgtttattgcagcttataatggttacaaataaagcaatagcatcacaaatttcacaaataaagcatttttttcactgc

SEQ ID NO: 19-20 - skipped (2 entries)

SEQ ID NO: 21 - RNA, 13 nt, synthetic construct
  source 1..13: mol_type = other RNA
gccgccaccatgg

SEQ ID NO: 22-23 - skipped (2 entries)

SEQ ID NO: 24 - DNA, 251 bp, synthetic construct
  source 1..251: mol_type = other DNA
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 25 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 26 - DNA, 395 bp, synthetic construct
  source 1..395: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtatttta

SEQ ID NO: 27 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

(19) United States
(12) Patent Application Publication
(10) Pub. No.: US 2025/0382637 A1
(43) Pub. Date: Dec. 18, 2025

(54) GENETIC REGULATORY ELEMENTS AND USES THEREOF

(71) Applicant: Regel Therapeutics, Inc., Cambridge, MA (US)

(72) Inventors: Jordane DIMIDSCHSTEIN, Boston, MA (US); Navneet MATHARU, San Francisco, CA (US); Kathryn ALLAWAY, Arlington, MA (US); Ryan ZIFFRA, Pleasant Hill, CA (US)

(21) Appl. No.: 18/876,697

(22) PCT Filed: Jun. 21, 2023
(86) PCT No.: PCT/US2023/068795

§ 371 Date: Dec. 19, 2024

(51) Int. Cl. C12N 15/86; C12N 9/22; C12N 15/11

(52) U.S. Cl. CPC C12N 15/86; C12N 9/226; C12N 15/11; C12N 2310/20; C12N 2750/14143; C12N 2750/14152; C12N 2830/48; C12N 2830/50; C12N 2840/203

(57) ABSTRACT

Provided herein are regulatory elements capable of restricting the expression of a heterologous nucleotide sequence to specific neuronal populations and regions of the central nervous system (CNS). Further provided herein are vectors comprising such regulatory elements and uses of such regulatory elements and vectors for selective expression in the CNS.

### CROSS-REFERENCE TO RELATED APPLICATIONS

This application claims priority to U.S. Provisional Patent Application No. 63/354,110, filed Jun. 21, 2022, which is incorporated by reference herein in its entirety for all purposes.

### REFERENCE TO AN ELECTRONIC SEQUENCE LISTING

The contents of the electronic sequence listing (REGT_002_01WO_SeqList_ST26.xml; Size: 31,745 bytes; and Date of Creation: Jun. 14, 2023) are herein incorporated by reference in their entirety).

### TECHNICAL FIELD

The disclosure relates to genetic regulatory elements and uses thereof to express heterologous nucleotide sequences.

### BACKGROUND

Gene therapy has the potential to revolutionize the treatment of genetic disorders. Clinical application of this promising technology requires overcoming significant technical limitations. Among these is the need to restrict the therapeutic intervention specifically to the cellular populations affected by the disease in order to reduce off-target effects and increase efficacy of the therapy.

### SUMMARY

Provided herein is a vector comprising (i) a heterologous nucleotide sequence; and (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 base pairs (bp) or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11. In some embodiments, the vector further comprises a promoter.

In some embodiments, the vector comprises, in 5′ to 3′ order: (i) the promoter; (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (iii) the heterologous nucleotide sequence.

In some embodiments, the vector comprises, in 5′ to 3′ order: (i) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (ii) the promoter; and (iii) the heterologous nucleotide sequence.

In some embodiments, the vector comprises, in 5′ to 3′ order: (i) the promoter; (ii) the heterologous nucleotide sequence; and (iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

In some embodiments, the vector is a non-viral vector. In some embodiments, the non-viral vector is a plasmid vector.

In some embodiments, the vector is a viral vector. In some embodiments, the viral vector is an adeno-associated virus (AAV) vector or an adenovirus vector.

In some embodiments, the AAV vector comprises a 5′ AAV inverted terminal repeat (ITR) and a 3′ AAV ITR flanking the heterologous nucleotide sequence, a promoter, and (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

In some embodiments, the AAV vector comprises, in 5′ to 3′ order: (i) the 5′ AAV ITR; (ii) the promoter; (iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (iv) the heterologous nucleotide sequence; and (v) the 3′ AAV ITR.

In some embodiments, the AAV vector comprises, in 5′ to 3′ order: (i) the 5′ AAV ITR; (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (iii) the promoter; (iv) the heterologous nucleotide sequence; and (v) the 3′ AAV ITR.

In some embodiments, the AAV vector comprises, in 5′ to 3′ order: (i) the 5′ AAV ITR; (ii) the promoter; (iii) the heterologous nucleotide sequence; (iv) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (v) the 3′ AAV ITR.

In some embodiments, the promoter comprises an RNA-polymerase II binding site, a TATA box, a TF2B recognition element (BRE), a motif 10 element (MTE), or a downstream promoter element (DPE).

In some embodiments, the promoter is a minimal promoter. In some embodiments, the minimal promoter is a human beta-globin minimal promoter.

In some embodiments, the vector further comprises an enhancer. In some embodiments, the vector further comprises or encodes a polyadenylation signal sequence. In some embodiments, the vector further comprises or encodes a Kozak sequence. In some embodiments, the vector further comprises or encodes a woodchuck hepatitis virus post-transcriptional element (WPRE), a hepatitis B virus posttranscriptional regulatory element, an RNA transport element (RTE), a WPRE3, or a wsl3 regulatory element. In some embodiments, the vector further comprises an artificial intron. In some embodiments, the vector further encodes a 2A self-cleaving peptide. In some embodiments, the vector further comprises or encodes an internal ribosome entry site (IRES).

In some embodiments, the heterologous nucleotide sequence encodes a protein. In some embodiments, the heterologous nucleotide sequence encodes a gene silencing nucleic acid. In some embodiments, the heterologous nucleotide sequence encodes a CRISPR-associated protein. In some embodiments, the heterologous nucleotide sequence further encodes a guide RNA (gRNA).

Provided herein is a viral particle comprising a vector disclosed herein. In some embodiments, the viral particle is a recombinant adenovirus particle. In some embodiments, the viral particle is a recombinant AAV (rAAV) particle. In some embodiments, the rAAV particle is an AAV1, AAV2, AAV3, AAV4, AAV5, AAV6, AAV7, AAV8, AAV9, AAV10, AAV11, AAV12, AAV13, AAVrh.8, AAVrh.10, AAVrh32.33, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHP.eB, AAV-CAP.B10, AAV2-r3.45, AAV2-LSS, AAV2PFG, AAV2-PPS, AAV2-TLH or AAV2-GMN serotype particle.

Provided herein is a population of viral particles comprising a plurality of viral particles disclosed herein.

Provided herein is a pharmaceutical composition comprising a vector, a viral particle, or a population disclosed herein, and a pharmaceutically acceptable carrier, vehicle or diluent.

Provided herein is a nanoparticle comprising a vector disclosed herein.

Provided herein is a cell comprising a vector or a viral particle disclosed herein. In some embodiments, the cell is a mammalian cell or an insect cell.

Provided herein is a method of producing a rAAV particle, the method comprising: (i) culturing a cell disclosed herein under conditions allowing for packaging the rAAV particle; and (ii) harvesting the cultured host cell or culture medium for collection of the rAAV particle.

Provided herein is a method for selectively expressing the heterologous nucleotide sequence in neurons of the central nervous system (CNS) in a subject in need thereof, the method comprising administering to the subject a vector, a viral particle, a population, a pharmaceutical composition, or a nanoparticle disclosed herein.

In some embodiments of the methods provided herein, the heterologous nucleotide sequence expression is enriched in specific regions of the CNS. In some embodiments, the heterologous nucleotide sequence expression is enriched in neurons of one or more of: the forebrain, the cerebellum, and the spinal cord. In some embodiments, the heterologous nucleotide sequence expression is enriched in neurons of the forebrain and the cerebellum. In some embodiments, the heterologous nucleotide sequence expression is enriched in neurons of the forebrain. In some embodiments, the heterologous nucleotide sequence expression is enriched in neurons of the cerebellum. In some embodiments, the heterologous nucleotide sequence expression is enriched in neurons of the cerebellum and hippocampus. In some embodiments, the heterologous nucleotide sequence expression is enriched in neurons of the spinal cord.

In some embodiments of the methods provided herein, the vector comprises the nucleotide sequence of SEQ ID NO:1 or SEQ ID NO: 10, and the heterologous nucleotide sequence expression is enriched in neurons of the CNS.

In some embodiments of the methods provided herein, the vector comprises the nucleotide sequence of SEQ ID NO: 2 or SEQ ID NO: 9, and the heterologous nucleotide sequence expression is enriched in neurons of the forebrain and the cerebellum.

In some embodiments of the methods provided herein, the vector comprises the nucleotide sequence of SEQ ID NO: 4, SEQ ID NO: 5, or SEQ ID NO: 11, and the heterologous nucleotide sequence expression is enriched in neurons of the forebrain.

In some embodiments of the methods provided herein, the vector comprises the nucleotide sequence of SEQ ID NO: 6, and the heterologous nucleotide sequence expression is enriched in neurons of the cerebellum and hippocampus.

In some embodiments of the methods provided herein, the vector, the viral particle, the population, the pharmaceutical composition, or the nanoparticle is administered to the subject intravenously. In some embodiments of the methods provided herein, the vector, the viral particle, the population, the pharmaceutical composition, or the nanoparticle is administered to the subject via injection into the CNS.

### BRIEF DESCRIPTION OF THE DRAWINGS

FIG. 1A depicts schematics and representative images of transgene expression profiles across organs. Expression profiles of the regulatory elements CNS2_V4 (SEQ ID NO: 1), CNS2_V3 (SEQ ID NO: 2), CNS2_V7 (SEQ ID NO: 4), and CNS2_V10 (SEQ ID NO: 6) (from top to bottom) in the indicated organs upon systemic injection of recombinant adeno-associated virus (rAAV) particles (having AAV-PHP.eB capsids) containing the regulatory elements into adult mice after 3 weeks of incubation are shown. Representative images show the expression of the viral reporter. n=3 mice per construct. Organs shown from left to right are: Sagittal IHC (immunohistochemistry); Cortex; Hippocampus; Cerebellum; Spinal Cord; DRG (dorsal root ganglion); Liver; Heart; Muscle; Lung.

FIG. 1B depicts schematics and representative images of transgene expression profiles across organs. Expression profiles of the regulatory elements CNS2_V4 (SEQ ID NO: 1), CNS2_V4.1 (SEQ ID NO: 10); CNS2_V3 (SEQ ID NO: 2), CNS2_V3.3 (SEQ ID NO: 9); CNS2_V7 (SEQ ID NO: 4), CNS2_V7.1 (SEQ ID NO: 11), CNS2_V11 (SEQ ID NO: 5), and CNS2_V10 (SEQ ID NO: 6) (from top to bottom) in the indicated organs upon systemic injection of rAAV particles (having AAV-PHP.eB capsids) containing the regulatory elements into adult mice after 3 weeks of incubation are shown. Representative images show the expression of the viral reporter. N=at least 3 mice per construct. Organs shown from left to right are: Sagittal IHC (immunohistochemistry); Cortex; Hippocampus; Striatum; Cerebellum; Spinal Cord; DRG (dorsal root ganglion); Liver; Heart; Muscle; Lung.

FIG. 2A-FIG. 2C depict results from quantitative analyses of intensity, specificity and sensitivity of transgene expression using regulatory elements CNS2_V3 (V3) (SEQ ID NO: 2), CNS2_V4 (V4) (SEQ ID NO: 1), and CNS2_V7 (V7) (SEQ ID NO: 4). Results are shown for neurons (expressing pan-neuronal marker NeuN) in the cortex, corresponding to the data presented in FIG. 1A. FIG. 2A depicts results of an analysis for specificity for neurons. FIG. 2B depicts results of an analysis for sensitivity for neurons. FIG. 2C depicts results of an analysis for sensitivity by cortical layer.

FIG. 3A-FIG. 3D depict results from quantitative analyses of intensity, specificity and sensitivity of transgene expression using regulatory elements CNS2_V4 (SEQ ID NO: 1), CNS2_V4.1 (SEQ ID NO: 10); CNS2_V3 (SEQ ID NO: 2), CNS2_V3.3 (SEQ ID NO: 9); CNS2_V7 (SEQ ID NO: 4), and CNS2_V7.1 (SEQ ID NO: 11). Results are shown for neurons (expressing pan-neuronal marker NeuN) in relevant regions, corresponding to the data presented in FIG. 1B. FIG. 3A depicts results of an analysis for specificity for neurons within the somatosensory cortex (ssctx). FIG. 3B depicts results of an analysis for sensitivity for neurons within the somatosensory cortex (ssctx). FIG. 3C depicts results of an analysis for sensitivity by cortical layer. FIG. 3D depicts results of an analysis for sensitivity for neurons within the striatum.

FIG. 4A-FIG. 4B depict results from a study of transgene expression after intracerebroventricular (ICV) injection of rAAV particles (having AAV9 capsids) containing the regulatory element CNS2_V4.1 (SEQ ID NO: 10) into postnatal day 1 (PND1) mice after 4 weeks of incubation. FIG. 4A depicts images of fluorescent reporter (dTomato) expression driven by CNS2_V4.1 within the brain. FIG. 4B depicts results of an analysis of dCas9 transgene expression driven by CNS2_V4.1 detected by qPCR.

### DETAILED DESCRIPTION

The disclosure provides regulatory elements capable of selectively expressing a heterologous nucleotide sequence in specific neuronal populations and regions of the central nervous system (CNS) while reducing or preventing expression of the heterologous nucleotide sequence in other organs. The regulatory elements enrich expression of the heterologous nucleotide sequence in neurons and reduce or prevent expression in other cellular populations (e.g., glia, microglia, and endothelial cells). When integrated into a DNA-based expression vector, the regulatory elements can be used to reduce off-target effects and increase efficacy of gene therapy. The regulatory elements show evidence of broad applicability, as delivery of the same regulatory element with different rAAV serotypes, in differently aged mice, and expressing different transgenes demonstrated the same expression profile.

### Regulatory Elements and Vectors

Provided herein are genetic regulatory elements for selectively expressing a heterologous nucleotide sequence in neurons of the CNS. When integrated in a DNA-based expression vector, these regulatory elements are capable of restricting the expression of the heterologous nucleotide sequence to neurons of the CNS. In some embodiments, the regulatory elements restrict the expression of the heterologous nucleotide sequence to neurons of the CNS with at least about 75%, about 80%, about 85%, about 90%, about 95%, or about 99% specificity for this cell type. In some embodiments, the regulatory elements restrict the expression of the heterologous nucleotide sequence to neurons of the CNS with at least about 75%, about 80%, about 85%, about 90%, about 95%, or about 99% selectivity for this cell type.

In some embodiments, the regulatory elements restrict the expression of the heterologous nucleotide sequence to neurons of the forebrain and cerebellum with at least about 75%, about 80%, about 85%, about 90%, about 95%, or about 99% specificity for this cell type. In some embodiments, the regulatory elements restrict the expression of the heterologous nucleotide sequence to neurons of the forebrain and cerebellum with at least about 75%, about 80%, about 85%, about 90%, about 95%, or about 99% selectivity for this cell type.

In some embodiments, the regulatory elements restrict the expression of the heterologous nucleotide sequence to neurons of the forebrain with at least about 75%, about 80%, about 85%, about 90%, about 95%, or about 99% specificity for this cell type. In some embodiments, the regulatory elements restrict the expression of the heterologous nucleotide sequence to neurons of the forebrain with at least about 75%, about 80%, about 85%, about 90%, about 95%, or about 99% selectivity for this cell type.

In some embodiments, the regulatory elements restrict the expression of the heterologous nucleotide sequence to neurons of the cerebellum with at least about 75%, about 80%, about 85%, about 90%, about 95%, or about 99% specificity for this cell type. In some embodiments, the regulatory elements restrict the expression of the heterologous nucleotide sequence to neurons of the cerebellum with at least about 75%, about 80%, about 85%, about 90%, about 95%, or about 99% selectivity for this cell type.

In some embodiments, the regulatory elements restrict the expression of the heterologous nucleotide sequence to neurons of the cerebellum and neurogenic niches with at least about 75%, about 80%, about 85%, about 90%, about 95%, or about 99% specificity for this cell type. In some embodiments, the regulatory elements restrict the expression of the heterologous nucleotide sequence to neurons of the cerebellum and neurogenic niches with at least about 75%, about 80%, about 85%, about 90%, about 95%, or about 99% selectivity for this cell type.

In some embodiments, the regulatory elements express the heterologous nucleotide sequence in non-neuronal cells of the CNS (e.g., glial cells, microglial cells or endothelial cells) with at most about 25%, about 20%, about 15%, about 10%, about 5%, or about 1% specificity for this cell type. In some embodiments, the regulatory elements express the heterologous nucleotide sequence in non-neuronal cells of the CNS (e.g., glial cells, microglial cells or endothelial cells) with at most about 25%, about 20%, about 15%, about 10%, about 5%, or about 1% selectivity for this cell type.

In some embodiments, the regulatory elements do not express the heterologous nucleotide sequence in one or more of liver, heart, muscle, and lung.

Provided herein is a genetic regulatory element comprising the nucleotide sequence of any of SEQ ID NOs: 1-13, a reverse nucleotide sequence of any of SEQ ID NOs: 1-13; a complementary nucleotide sequence of any of SEQ ID NOs: 1-13; or a reverse complementary sequence of any of SEQ ID NOs: 1-13.

Provided herein is a genetic regulatory element comprising the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11, a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or a reverse complementary sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

Provided herein is a vector (e.g., a DNA vector) comprising (i) a heterologous nucleotide sequence; and (ii) the nucleotide sequence of any of SEQ ID NOs: 1-13; a reverse nucleotide sequence of any of SEQ ID NOs: 1-13; a complementary nucleotide sequence of any of SEQ ID NOs: 1-13; or a reverse complementary sequence of any of SEQ ID NOs: 1-13.

Provided herein is a vector (e.g., a DNA vector) comprising (i) a heterologous nucleotide sequence; and (ii) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or a reverse complementary sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

In some embodiments, a vector comprises (i) a heterologous nucleotide sequence; and (ii) a nucleotide sequence comprising one or more regions of about 100 base pairs (bp) or longer having at least 75%, 80%, 85%, 90%, 95%, 97%, or 99% or greater sequence identity to any of SEQ ID NOs: 1-13.

In some embodiments, a vector comprises (i) a heterologous nucleotide sequence; and (ii) a nucleotide sequence comprising one or more regions of about 100 base pairs (bp) or longer having at least 75%, 80%, 85%, 90%, 95%, 97%, or 99% or greater sequence identity to any of SEQ ID NOs: SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

In some embodiments, a vector comprises 2, 3, 4, 5, 6, 7, 8, 9, 10, 11, 12, or 13 of the regulatory elements disclosed herein. In some embodiments, a vector comprises 2, 3, 4, 5, 6, 7, or 8 of the regulatory elements CNS2_V4 (SEQ ID NO: 1), CNS2_V4.1 (SEQ ID NO: 10); CNS2_V3 (SEQ ID NO: 2), CNS2_V3.3 (SEQ ID NO: 9); CNS2_V7 (SEQ ID NO: 4), CNS2_V7.1 (SEQ ID NO: 11), CNS2_V11 (SEQ ID NO: 5), and CNS2_V10 (SEQ ID NO: 6).

In some embodiments, a vector provided herein further comprises a promoter. In some embodiments, the promoter is a minimal promoter. In some embodiments, a minimal promoter comprises one or more short sequences capable of promoting transcription. In some embodiments, a promoter comprises an RNA-polymerase II binding site, a TATA box, a TF2B recognition element (BRE), a motif 10 element (MTE), or a downstream promoter element (DPE). A TATA transcriptional regulatory activation site is described in e.g., François et al., (2005) J. Virol. 79(17):11082-11094.

In some embodiments, the promoter is a human beta-globin minimal promoter.

In some embodiments, a promoter is recognized by RNA polymerase II. In some embodiments, a promoter is recognized by RNA polymerase III. In some embodiments, a promoter is a human U6 (hU6) promoter, a mouse U6 promoter or a human H1 promoter.

In some embodiments, a promoter is a constitutive promoter. In some embodiments, a promoter is an inducible promoter. In some embodiments, a promoter is a tissue-specific promoter. In some embodiments, a promoter is the chicken beta-actin (CBA) promoter, the GUSB240 promoter, the GUSB379 promoter, the HSVTK promoter, the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the EF-1 alpha promoter, the dihydrofolate reductase (DHFR) promoter or the phosphoglycerol kinase (PGK) promoters.

TABLE 1
Exemplary regulatory sequences

| SEQ | | Size | |
|---|---|---|---|
| ID | | (base | |
| NO | Name | pairs) | Sequence |
| 1 | CNS2_V4 | 447 | cagcactgaaggcgcgctgacgtcactcgccggtcccccgcaaactccccttcccggccaccttggtcg |
| | | | cgtccgcgccgccgccggcccagccggaccgcaccacgcgaggcgcgagataggggggcaccagcactg |
| | | | aaggcgcgctgacgtcactcgccggtcccccgcaaactccccttcccggccaccttggtcgcgtccgcg |
| | | | ccgccgccggcccagccggaccgcaccacgcgaggcgcgagataggggggcaccagcactgaaggcgcg |
| | | | ctgacgtcactcgccggtcccccgcaaactccccttcccggccaccttggtcgcgtccgcgccgccgcc |
| | | | ggcccagccggaccgcaccacgcgaggcgcgagataggggggcacagctcgggctgggcataaaagtca |
| | | | gggcagagccatctattgcttacatttgcttct |
| 2 | CNS2_V3 | 537 | cccgacccactggacaagcacccaacccccattccccaaattgcgcatcccctatcagagagggggagg |
| | | | ggaaacaggatgcggcgaggcgcgtgcgcactgccagcttcagcaccgcggacagtgccttcgcccccg |
| | | | cctggcggcgcgcgccaccgcccccgacccactggacaagcacccaacccccattccccaaattgcgca |
| | | | tcccctatcagagagggggaggggaaacaggatgcggcgaggcgcgtgcgcactgccagcttcagcacc |
| | | | gcggacagtgccttcgcccccgcctggcggcgcgcgccaccgcccccgacccactggacaagcacccaa |
| | | | cccccattccccaaattgcgcatcccctatcagagagggggaggggaaacaggatgcggcgaggcgcgt |
| | | | gcgcactgccagcttcagcaccgcggacagtgccttcgcccccgcctggcggcgcgcgccaccgccagc |
| | | | tcgggctgggcataaaagtcagggcagagccatctattgcttacatttgcttct |
| 3 | CNS2_V2 | 217 | cccgacccactggacaagcacccaacccccattccccaaattgcgcatcccctatcagagagggggagg |
| | | | ggaaacaggatgcggcgaggcgcgtgcgcactgccagcttcagcaccgcggacagtgccttcgcccccg |
| | | | cctggcggcgcgcgccaccgccagctcgggctgggcataaaagtcagggcagagccatctattgcttac |
| | | | atttgcttct |
| 4 | CNS2_V7 | 624 | ggaagggaaggagagatgaattagcttcccctgtaaaccttagaacccagctgttgccagggcaacggg |
| | | | gcaatacctgtctcttcagaggagatgaagttgccagggtaactacatcctgtctttctcaaggaccat |
| | | | cccagaatgtggcacccactagccgttaccatagcaactgcctctttgccccacttaatcccatcccgt |
| | | | ctgttaaaagggccctatagttggaggtgggggaggtaggaagagcgatgatcacttgtggactaagtt |
| | | | tgttcgcatccccttctccaaccccctcagtacatcaccctgggggaacagggtccacttgctcctggg |
| | | | cccacacagtcctgcagtattgtgtatataaggccagggcaaagaggagcaggttttaaagtgaaaggc |
| | | | aggcaggtgttggggaggcagttaccggggcaacgggaacagggcgtttcggaggtggttgccatgggg |
| | | | acctggatgctgacgaaggctcgcgaggctgtgagcagccacagtgccctgctcagaagccccaagctc |
| | | | gtcagtcaagccggttctccgtttgcactcaggagcacgggcaggcgagtggcccctagttctgggggc |
| | | | agc |
| 5 | CNS2_V11 | 531 | gggcttctccatctgtctgggttagtacctgccactatactggaataaggggacgcctgcttccctcga |
| | | | gttggctggacaaggttatgagcatccgtgtacttatggggttgccagcttggtcctggatcgcccggg |
| | | | cccttcccccacccgttcggttccccaccaccacccgcgctcgtacgtgcgtctccgcctgcagctctt |
| | | | gactcatcggggccccccgggtcacatgcgctcgctcggctctataggcgccgccccctgcccaccccc |
| | | | cgcccgcgctgggagccgcagccgccgccactcctgctctctctgcgccgccgccgtcaccaccgccac |
| | | | cgccaccggctgagtctgcagtcctcgaggtgaggcccgtatcgggccgcatccccttactctgtcctt |
| | | | gccgcgtccactccccgttatcctgggcttagcgcccccgggtgtgtgtgtgtgtgtgtgtgtgtgtgt |
| | | | gtgtgtgtgtgtgtgtgtgtgtgtgtgtgtgacgctgcgtacctgtca |
| 6 | CNS2_V10 | 452 | tcccgcaaccccccccccccccacatgtcctctgtcttctgctgccacaaagggttaatctctcctgcg |
| | | | ggtaaaaacaggtccgcggagtctctaactggcgacagatgggccactttcttctggccacaaaggggc |
| | | | cggaatggagcgctccgcggcatacaaataggcaggtcacgtggttcccggctcttggctggaccggga |
| | | | agaccatatggcgcatgccggggaggaaggagcaggggcggaggtactgtgggggtgaggggagtggtg |
| | | | gtggaagggggcgggaggaaagttctggggaggggtgaatgagggcgggagtcgttcagtctggacgcg |
| | | | tgcgcgattgcggggcgcagggacctgctcgttgctcagctcacggccccgcccgcgctcagcatcagc |
| | | | aactcggctatataaccctagcgcccgcgccaccggga |
| 7 | CNS2_V3.1 | 629 | cccgacccactggacaagcacccaacccccattccccaaattgcgcatcccctatcagagagggggagg |
| | | | ggaaacaggatgcggcgaggcgcgtgcgcactgccagcttcagcaccgcggacagtgccttcgcccccg |
| | | | cctggcggcgcgcgccaccgcccccgacccactggacaagcacccaacccccattccccaaattgcgca |
| | | | tcccctatcagagagggggaggggaaacaggatgcggcgaggcgcgtgcgcactgccagcttcagcacc |
| | | | gcggacagtgccttcgcccccgcctggcggcgcgcgccaccgcccccgacccactggacaagcacccaa |
| | | | cccccattccccaaattgcgcatcccctatcagagagggggaggggaaacaggatgcggcgaggcgcgt |
| | | | gcgcactgccagcttcagcaccgcggacagtgccttcgcccccgcctggcggcgcgcgccaccgccagc |
| | | | tcgggctgggcataaaagtcagggcagagccatctattgcttacatttgcttctAAGAGGTAAGGGTTT |
| | | | AAGGGATGGTTGGTTGGTGGGGTATTAATGTTTAATTACCTGGAGCACCTGCCTGAAATCACTTTTTTT |
| | | | CAGGTTGG |
| 8 | CNS2_V3.2 | 617 | acccaacccccattccccaaattgcgcatcccctatcagagagggggaggggaaacaggatgcggcgag |
| | | | gcgcgtgcgcactgccagcttcagcaccgcggacagtgccttcgcccccgcctggcggcgcgcgccacc |
| | | | gcacccaacccccattccccaaattgcgcatcccctatcagagagggggaggggaaacaggatgcggcg |
| | | | aggcgcgtgcgcactgccagcttcagcaccgcggacagtgccttcgcccccgcctggcggcgcgcgcca |
| | | | ccgcacccaacccccattccccaaattgcgcatcccctatcagagagggggaggggaaacaggatgcgg |
| | | | cgaggcgcgtgcgcactgccagcttcagcaccgcggacagtgccttcgcccccgcctggcggcgcgcgc |
| | | | caccgcacccaacccccattccccaaattgcgcatcccctatcagagagggggaggggaaacaggatgc |
| | | | ggcgaggcgcgtgcgcactgccagcttcagcaccgcggacagtgccttcgcccccgcctggcggcgcgc |
| | | | gccaccgcagctcgggctgggcataaaagtcagggcagagccatctattgcttacatttgcttct |
| 9 | CNS2_V3.3 | 587 | atcagagagggggaggggaaacaggatgcggcgaggcgcgtgcgcactgccagcttcagcaccgcggac |
| | | | agtgccttcgcccccgcctggcggcgcgcgccaccgcatcagagagggggaggggaaacaggatgcggc |
| | | | gaggcgcgtgcgcactgccagcttcagcaccgcggacagtgccttcgcccccgcctggcggcgcgcgcc |
| | | | accgcatcagagagggggaggggaaacaggatgcggcgaggcgcgtgcgcactgccagcttcagcaccg |
| | | | cggacagtgccttcgcccccgcctggcggcgcgcgccaccgcatcagagagggggaggggaaacaggat |
| | | | gcggcgaggcgcgtgcgcactgccagcttcagcaccgcggacagtgccttcgcccccgcctggcggcgc |
| | | | gcgccaccgcatcagagagggggaggggaaacaggatgcggcgaggcgcgtgcgcactgccagcttcag |
| | | | caccgcggacagtgccttcgcccccgcctggcggcgcgcgccaccgcagctcgggctgggcataaaagt |
| | | | cagggcagagccatctattgcttacatttgcttct |
| 10 | CNS2_V4.1 | 537 | gaaggcgcgctgacgtcactcgccggtcccccgcaaactccccttcccggccaccttggtcgcgtccgc |
| | | | gccgccgccgggaaggcgcgctgacgtcactcgccggtcccccgcaaactccccttcccggccaccttg |
| | | | gtcgcgtccgcgccgccgccgggaaggcgcgctgacgtcactcgccggtcccccgcaaactccccttcc |
| | | | cggccaccttggtcgcgtccgcgccgccgccgggaaggcgcgctgacgtcactcgccggtcccccgcaa |
| | | | actccccttcccggccaccttggtcgcgtccgcgccgccgccgggaaggcgcgctgacgtcactcgccg |
| | | | gtcccccgcaaactccccttcccggccaccttggtcgcgtccgcgccgccgccgggaaggcgcgctgac |
| | | | gtcactcgccggtcccccgcaaactccccttcccggccaccttggtcgcgtccgcgccgccgccggagc |
| | | | tcgggctgggcataaaagtcagggcagagccatctattgcttacatttgcttct |
| 11 | CNS2_V7.1 | 523 | ggaagggaaggagagatgaattagcttcccctgtaaaccttagaacccagctgttgccagggcaacggg |
| | | | gcaatacctgtctcttcagaggagatgaagttgccagggtaactacatcctgtctttctcaaggaccat |
| | | | cccagaatgtggcacccactagccgttaccatagcaactgcctctttgccccacttaatcccatcccgt |
| | | | ctgttaaaagggccctatagttggaggtgggggaggtaggaagagcgatgatcacttgtggactaagtt |
| | | | tgttcgcatccccttctccaaccccctcagtacatcaccctgggggaacagggtccacttgctcctggg |
| | | | cccacacagtcctgcagtattgtgtatataaggccagggcaaagaggagcaggttttaaagtgaaaggc |
| | | | aggcaggtgttggggaggcagttaccggggcaacgggaacagggcgtttcggaggtggttgccatgggg |
| | | | acctggatgctgacgaaggctcgcgaggctgtgagcagcc |
| 12 | CNS2_V7.2 | 627 | gaagggaaggagagatgaattagcttcccctgtaaaccttagaacccagctgttgccagggcaacgggg |
| | | | caatacctgtctcttcagaggagatgaagttgccagggtaactacatcctgtctttctcaaggaccatc |
| | | | ccagaatgtggcacccactagccgttaccatagcaactgcctctttgccccacttaatcccatcccgtc |
| | | | tgttaaaagggccctatagttggaggtgggggaggtaggaagagcgatgatcacttgtggactaagttt |
| | | | gttcgcatcgaagggaaggagagatgaattagcttcccctgtaaaccttagaacccagctgttgccagg |
| | | | gcaacggggcaatacctgtctcttcagaggagatgaagttgccagggtaactacatcctgtctttctca |
| | | | aggaccatcccagaatgtggcacccactagccgttaccatagcaactgcctctttgccccacttaatcc |
| | | | catcccgtctgttaaaagggccctatagttggaggtgggggaggtaggaagagcgatgatcacttgtgg |
| | | | actaagtttgttcgcatcagctcgggctgggcataaaagtcagggcagagccatctattgcttacattt |
| | | | gcttct |
| 13 | CNS2_V7.3 | 525 | acacagtcctgcagtattgtgtatataaggccagggcaaagaggagcaggttttaaagtgaaaggcagg |
| | | | caggtgttggggaggcagttaccggggcaacgggaacagggcgtttcggaggtggttgccatggggacc |
| | | | tggatgctgacgaaggctcgcgaggctgtgagcagccacacagtcctgcagtattgtgtatataaggcc |
| | | | agggcaaagaggagcaggttttaaagtgaaaggcaggcaggtgttggggaggcagttaccggggcaacg |
| | | | ggaacagggcgtttcggaggtggttgccatggggacctggatgctgacgaaggctcgcgaggctgtgag |
| | | | cagccacacagtcctgcagtattgtgtatataaggccagggcaaagaggagcaggttttaaagtgaaag |
| | | | gcaggcaggtgttggggaggcagttaccggggcaacgggaacagggcgtttcggaggtggttgccatgg |
| | | | ggacctggatgctgacgaaggctcgcgaggctgtgagcagcc |
| 14 | CNS2_V5 | 88 | ggcggggcccggtcgcccctccccctccccgccctcctgcgccgggagcagtgcattgtgggaaactcc |
| | | | cgagctctcctccgcgttc |
| 15 | CNS2_V6 | 252 | ggcccggtcgcccctccccctccccgccctcctgcgccgggagcagtgcattgtgggaaactcccggcc |
| | | | cggtcgcccctccccctccccgccctcctgcgccgggagcagtgcattgtgggaaactcccggcccggt |
| | | | cgcccctccccctccccgccctcctgcgccgggagcagtgcattgtgggaaactcccagctcgggctgg |
| | | | gcataaaagtcagggcagagccatctattgcttacatttgcttct |
| 16 | CNS2_V8 | 733 | tgacaatgctccctctggcctcactcccttttatacgtacttccatcaaaccatctgattcaacaatga |
| | | | cagaccgatctcttatgggcttggcacacaccatctgcccattataaacgtctgcaaagaccaaggttt |
| | | | gatatgttgattttactgtcagccttaagagtgcgacatctgctaatttagtgtaataatacaatcagt |
| | | | agaccctttaaaacaagtcccttggcttggaacaacgccaggctcctcaacaggcaactttgctacttc |
| | | | tacagaaaatgataataaagaaatgctggtgaagtcaaatgcttatcacaatggtgaactactcagcag |
| | | | ggaggctctaataggcgccaagagcctagacttccttaagcgccagagtccacaagggcccagttaatc |
| | | | ctcaacattcaaatgctgcccacaaaaccagcccctctgtgccctagccgcctcttttttccaagtgac |
| | | | agtagaactccaccaatccgcagctgaatggggtccgcctcttttccctgcctaaacagacaggaactc |
| | | | ctgccaattgagggcgtcaccgctaaggctccgccccagcctgggctccacaaccaatgaagggtaatc |
| | | | tcgacaaagagcaaggggtggggcgcgggcgcgcaggtgcagcagcacacaggctggtcgggagggcgg |
| | | | ggcgcgacgtctgccgtgcggggtcccggcatcggttgcgcgc |
| 17 | CNS2_V9 | 361 | agacttccttaagcgccagagtccacaagggcccagttaatcctcaacattcaaatgctgcccacaaaa |
| | | | ccagcccctctgtgccctagccgcctcttttttccaagtgacagtagaactccaccaatccgcagctga |
| | | | atggggtccgcctcttttccctgcctaaacagacaggaactcctgccaattgagggcgtcaccgctaag |
| | | | gctccgccccagcctgggctccacaaccaatgaagggtaatctcgacaaagagcaaggggtggggcgcg |
| | | | ggcgcgcaggtgcagcagcacacaggctggtcgggagggcggggcgcgacgtctgccgtgcggggtccc |
| | | | ggcatcggttgcgcgc |

TABLE 2
Expression profiles of exemplary regulatory sequences

| SEQ | | |
|---|---|---|
| ID NO | Name | Expression profile |
| 1 | CNS2_V4 | Pan-neuronal expression across all brain regions |
| 2 | CNS2_V3 | Forebrain and cerebellum-enriched expression |
| | | (neuron-specific) |
| 3 | CNS2_V2 | Neuron-specific expression |
| 4 | CNS2_V7 | Forebrain-enriched expression (neuron-specific) |
| 5 | CNS2_V11 | Forebrain-enriched expression (neuron-specific) |
| 6 | CNS2_V10 | Cerebellum and hippocampus-enriched |
| | | expression (neuron-specific) |
| 7 | CNS2_V3.1 | Forebrain and cerebellum-enriched expression |
| | | (neuron-specific) |
| 8 | CNS2_V3.2 | Forebrain and cerebellum-enriched expression |
| | | (neuron-specific) |
| 9 | CNS2_V3.3 | Forebrain and cerebellum-enriched expression |
| | | (neuron-specific) |
| 10 | CNS2_V4.1 | Pan-neuronal expression across all brain regions |
| 11 | CNS2_V7.1 | Forebrain-enriched expression (neuron-specific) |
| 12 | CNS2_V7.2 | |
| 13 | CNS2_V7.3 | Forebrain-enriched expression (neuron-specific) |
| 14 | CNS2_V5 | |
| 15 | CNS2_V6 | |
| 16 | CNS2_V8 | Neuron-specific expression |
| 17 | CNS2_V9 | Neuron-specific expression |

In some embodiments, a vector provided herein comprises (a) the promoter; (b) the nucleotide sequence of any of SEQ ID NOs: 1-13; a reverse nucleotide sequence of any of SEQ ID NOs: 1-13; a complementary nucleotide sequence of any of SEQ ID NOs: 1-13; a reverse complementary sequence of any of SEQ ID NOs: 1-13; or a nucleotide sequence comprising one or more regions of about 100 bp or longer having at least 75% or greater sequence identity to any of SEQ ID NOs: 1-13; and (c) the heterologous nucleotide sequence.

In some embodiments, a vector provided herein comprises (a) the promoter; (b) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a reverse complementary sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (c) the heterologous nucleotide sequence.

In some embodiments, a vector provided herein comprises, in 5′ to 3′ order: (i) the promoter; (ii) (a) the nucleotide sequence of any of SEQ ID NOs: 1-13; (b) a reverse nucleotide sequence of any of SEQ ID NOs: 1-13; (c) a complementary nucleotide sequence of any of SEQ ID NOs: 1-13; (d) a reverse complementary nucleotide sequence of any of SEQ ID NOs: 1-13; or (e) a nucleotide sequence comprising one or more regions of about 100 bp or longer having at least 75% or greater sequence identity to any of SEQ ID NOs: 1-13; and (iii) the heterologous nucleotide sequence.

In some embodiments, a vector provided herein comprises, in 5′ to 3′ order: (i) the promoter; (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (iii) the heterologous nucleotide sequence.

In some embodiments, a vector provided herein comprises, in 5′ to 3′ order: (i) (a) the nucleotide sequence of any of SEQ ID NOs: 1-13; (b) a reverse nucleotide sequence of any of SEQ ID NOs: 1-13; (c) a complementary nucleotide sequence of any of SEQ ID NOs: 1-13; (d) a reverse complementary nucleotide sequence of any of SEQ ID NOs: 1-13; or (e) a nucleotide sequence comprising one or more regions of about 100 bp or longer having at least 75% or greater sequence identity to any of SEQ ID NOs: 1-13; and (ii) the promoter; and (iii) the heterologous nucleotide sequence.

In some embodiments, a vector provided herein comprises, in 5′ to 3′ order: (i) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (ii) the promoter; and (iii) the heterologous nucleotide sequence.

In some embodiments, a vector provided herein comprises, in 5′ to 3′ order: (i) the promoter; (ii) the heterologous nucleotide sequence; and (iii) (a) the nucleotide sequence of any of SEQ ID NOs: 1-13; (b) a reverse nucleotide sequence of any of SEQ ID NOs: 1-13; (c) a complementary nucleotide sequence of any of SEQ ID NOs: 1-13; (d) a reverse complementary nucleotide sequence of any of SEQ ID NOs: 1-13; or (e) a nucleotide sequence comprising one or more regions of about 100 bp or longer having at least 75% or greater sequence identity to any of SEQ ID NOs: 1-13.

In some embodiments, a vector provided herein comprises, in 5′ to 3′ order: (i) the promoter; (ii) the heterologous nucleotide sequence; and (iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

In some embodiments, a vector provided herein further comprises an enhancer.

In some embodiments, a vector provided herein further comprises or encodes a polyadenylation (polyA) signal sequence. As used herein, a “polyadenylation signal sequence” refers to a DNA sequence that when transcribed regulates the addition of a polyA tail to the mRNA transcript. In some embodiments, a polyA signal sequence is a SV40, human, bovine or rabbit polyA signal sequence. In some embodiments, a polyA signal sequence is a SV40 polyA signal sequence. In some embodiments, a polyA signal sequence is a β-globin polyA signal sequence. In some embodiments, a polyA signal sequence is a human growth hormone polyA signal sequence or a bovine growth hormone polyA signal sequence.

In some embodiments, a SV40 polyA signal sequence comprises or consists of

(SEQ ID NO: 18)
AACTTGTTTATTGCAGCTTATAATGGTTACAAATAAAGCAATAGCATCA
CAAATTTCACAAATAAAGCATTTTTTTCACTGC.

In some embodiments, a human growth hormone polyadenylation signal (bGHpA) sequence comprises or consists of

(SEQ ID NO: 25)
Gggtggcatccctgtgacccctccccagtgcctctcctggccctggaag
ttgccactccagtgcccaccagccttgtcctaataaaattaagttgcat
cattttgtctgactaggtgtccttctataatattatggggtggaggggg
gtggtatggagcaaggggcaagttgggaagacaacctgtagggcctgcg
gggtctattgggaaccaagctggagtgcagtggcacaatcttggctcac
tgcaatctccgcctcctgggttcaagcgattctcctgcctcagcctccc
gagttgttgggattccaggcatgcatgaccaggctcagctaatttttgt
ttttttggtagagacggggtttcaccatattggccaggctggtctccaa
ctcctaatctcaggtgatctacccaccttggcctcccaaattgctggga
ttacaggcgtgaaccactgctcccttccctgtcctt.

In some embodiments, a human β-globin polyadenylation signal (hBGpA) sequence comprises or consists of

(SEQ ID NO: 26)
Gctcgctttcttgctgtccaatttctattaaaggttcctttgttcccta
agtccaactactaaactgggggatattatgaagggccttgagcatctgg
attctgcctaataaaaaacatttattttcattgcaatgatgtatttaaa
ttatttctgaatattttactaaaaagggaatgtgggaggtcagtgcatt
taaaacataaagaaatgaagagctagttcaaaccttgggaaaatacact
atatcttaaactccatgaaagaaggtgaggctgcaaacagctaatgcac
attggcaacagcccctgatgcctatgccttattcatccctcagaaaagg
attcaagtagaggcttgatttggaggttaaagttttgctatgctgtatt
tta.

In some embodiments, a vector provided herein further comprises or encodes a Kozak sequence (for example, a DNA sequence transcribed to an RNA Kozak sequence). In some embodiments, a vector comprises a Kozak sequence upstream of the transgene. In some embodiments, the Kozak sequence is encoded by GCCACC (SEQ ID NO: 19). In some embodiments, the Kozak sequence (e.g., RNA Kozak sequence) comprises or consists of ACCAUGG (SEQ ID NO: 20), GCCGCCACCAUGG (SEQ ID NO: 21), CCACCAUG (SEQ ID NO: 22) or CCACCAUGG (SEQ ID NO: 23).

In some embodiments, a vector provided herein further comprises or encodes a woodchuck hepatitis virus post-transcriptional element (WPRE). See, e.g., Wang and Verma, Proc. Natl. Acad. Sci., USA, 96: 3906-3910 (1999). In some embodiments, a vector comprises or encodes a hepatitis B virus posttranscriptional regulatory element (HBVPRE) and/or an RNA transport element (RTE). In some embodiments, the WPRE or HBVPRE sequence is any of the WPRE or HBVPRE sequences disclosed in U.S. Pat. No. 6,136,597 or U.S. Pat. No. 6,287,814. In some embodiments, a vector provided herein further comprises or encodes a WPRE3 or a wsl3 regulatory element.

In some embodiments a WPRE3 comprises or is encoded b

(SEQ ID NO: 24)
GATAATCAACCTCTGGATTACAAAATTTGTGAAAGATTGACTGGTATTC
TTAACTATGTTGCTCCTTTTACGCTATGTGGATACGCTGCTTTAATGCC
TTTGTATCATGCTATTGCTTCCCGTATGGCTTTCATTTTCTCCTCCTTG
TATAAATCCTGGTTAGTTCTTGCCACGGCGGAACTCATCGCCGCCTGCC
TTGCCCGCTGCTGGACAGGGGCTCGGCTGTTGGGCACTGACAATTCCGT
GGTGTT.

In some embodiments, a WPRE comprises or is encoded by

(SEQ ID NO: 27)
AATCAACCTCTGGATTACAAAATTTGTGAAAGATTGACTGGTATTCTTA
ACTATGTTGCTCCTTTTACGCTATGTGGATACGCTGCTTTAATGCCTTT
GTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTG
GGGCATTGCCACCACCTGTCAGCTCCTTTCCGGGACTTTCGCTTTCCCC
CTCCCTATTGCCACGGCGGAACTCATCGCCGCCTGCCTTGCCCGCTGCT
GGACAGGGGCTCGGCTGTTGGGCACTGACAATTCCGTGGTGTTGTCGGG
GAAATCATCGTCCTTTCCTTGGCTGCTCGCCTGTGTTGCCACCTGGATT
CTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCG
C.

In some embodiments, a vector provided herein further comprises an artificial intron. In some embodiments, a vector provided herein further comprises a chimeric intron.

In some embodiments, a vector provided herein further encodes a 2A self-cleaving peptide.

In some embodiments, a vector provided herein further comprises or encodes an internal ribosome entry site (IRES). In some embodiments, an IRES is located on the 5′ end of the gene of interest and enables the translation of RNA in a cap-independent manner. The transcribed IRES may directly bind to a ribosomal subunit, such that the initiation codon of an mRNA is appropriately oriented in a ribosome to perform translation. The IRES sequence is usually located in the 5′ UTR of an mRNA (directly upstream of the initiation codon). An IRES functionally replaces the need for various protein factors that interact with the translation mechanism of eukaryotes.

Provided herein is a vector comprising (a) the promoter; (b) the nucleotide sequence of any of SEQ ID NOs: 1-13, or a reverse complementary sequence of any of SEQ ID NOs: 1-13; and (c) the heterologous nucleotide sequence; and comprising or encoding one or more of the following: an enhancer, a polyadenylation signal sequence, a Kozak sequence, a WPRE, a hepatitis B virus posttranscriptional regulatory element, a RTE, a WPRE3, a wsl3 regulatory element, an artificial intron, a chimeric intron, a 2A self-cleaving peptide, and an IRES.

Provided herein is a vector comprising (a) the promoter; (b) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11, or a reverse complementary sequence of any of SEQ ID NOs: SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (c) the heterologous nucleotide sequence; and comprising or encoding one or more of the following: an enhancer, a polyadenylation signal sequence, a Kozak sequence, a WPRE, a hepatitis B virus posttranscriptional regulatory element, a RTE, a WPRE3, a wsl3 regulatory element, an artificial intron, a chimeric intron, a 2A self-cleaving peptide, and an IRES.

In some embodiments, a vector provided herein further encodes nuclear localization signals (NLSs). In some embodiments, a NLS is a simian virus 40 (SV40) NLS. In some embodiments, a NLS is a nucleoplasmin NLS.

The genetic regulatory elements provided herein can be used to restrict a gene therapy intervention (e.g., gene replacement, gene modulation, gene editing, and/or modulation of cellular activity) to distinct neuronal populations in the CNS while preventing or reducing the effect of the gene therapy intervention in any other organs.

In some embodiments, the heterologous nucleotide sequence encodes a protein.

In some embodiments, the heterologous nucleotide sequence encodes a gene silencing nucleic acid. In some embodiments, the gene silencing nucleic acid is a short-hairpin RNA (shRNA). In some embodiments, the gene silencing nucleic acid is a short interfering RNA (siRNA).

In some embodiments, the heterologous nucleotide sequence encodes a CRISPR-associated protein. In some embodiments, the heterologous nucleotide sequence encodes a nuclease-deficient CRISPR-associated protein (also known as a catalytically inactive CRISPR nuclease, a dead Cas protein, or a dCas protein). In some embodiments, the heterologous nucleotide sequence encodes a CRISPR-associated protein and a guide RNA (gRNA).

In some embodiments, the heterologous nucleotide sequence encodes a transcription activator-like protein effector (TALE) protein.

In some embodiments, the vectors provided herein are plasmids or viral expression cassettes that comprise additional nucleic acid sequences. In some embodiments, the vectors provided herein may be used to generate recombinant virus particles to serve as viral vectors for gene delivery. In some embodiments, the vectors provided herein are formulated for use with via non-viral delivery systems. Further provided herein are plasmids comprising any of the vector nucleic acid sequences disclosed herein.

In some embodiments, a vector provided herein is a non-viral vector. In some embodiments, the vector is a plasmid vector. In some embodiments, the vector is a bacterial plasmid vector. In some embodiments, the vector is a yeast plasmid vector.

In some embodiments, the vector is a bacterial artificial chromosome vector, a yeast plasmid vector, or a yeast mini-chromosome vector.

In some embodiments, a vector provided herein is a viral vector.

In some embodiments, the viral vector is an adenovirus vector. Adenovirus vectors and recombinant adenoviruses are described in, for example, U.S. Pat. No. 10,781,427. In some embodiments, an adenovirus vector is a helper-dependent adenovirus vector that lacks all viral genes except the W packaging and inverted terminal repeat (ITR) sequences.

In some embodiments, the viral vector is an adeno-associated virus (AAV) vector.

In some embodiments, an AAV vector provided herein is self-complementary. In some embodiments, an AAV vector provided herein is single-stranded.

In some embodiments, an AAV vector provided herein comprises a first AAV ITR located upstream of the heterologous nucleotide sequence, promoter, and the nucleotide sequence of any of SEQ ID NOs: 1-13; a reverse nucleotide sequence of any of SEQ ID NOs: 1-13; a complementary nucleotide sequence of any of SEQ ID NOs: 1-13; a reverse complementary sequence of any of SEQ ID NOs: 1-13; or a nucleotide sequence comprising one or more regions of about 100 bp or longer having at least 75% or greater sequence identity to any of SEQ ID NOs: 1-13; and a second AAV ITR located downstream of the heterologous nucleotide sequence, promoter, and the nucleotide sequence of any of SEQ ID NOs: 1-13; a reverse nucleotide sequence of any of SEQ ID NOs: 1-13; a complementary nucleotide sequence of any of SEQ ID NOs: 1-13; a reverse complementary sequence of any of SEQ ID NOs: 1-13; or a nucleotide sequence comprising one or more regions of about 100 bp or longer having at least 75% or greater sequence identity to any of SEQ ID NOs: 1-13. ITRs are sequences that mediate AAV proviral integration and packaging of AAV DNA into virions.

In some embodiments, an AAV vector provided herein comprises a first AAV ITR located upstream of the heterologous nucleotide sequence, promoter, and the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a reverse complementary sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and a second AAV ITR located downstream of the heterologous nucleotide sequence, promoter, and the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; a complementary nucleotide sequence of any of SEQ ID NOs: 1-13; a reverse complementary sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

In some embodiments, an AAV vector provided herein comprises a 5′ AAV inverted terminal repeat (ITR) and a 3′ AAV ITR flanking the heterologous nucleotide sequence, a promoter, and (a) the nucleotide sequence of any of SEQ ID NOs: 1-13; (b) a reverse nucleotide sequence of any of SEQ ID NOs: 1-13; (c) a complementary nucleotide sequence of any of SEQ ID NOs: 1-13; (d) a reverse complementary nucleotide sequence of any of SEQ ID NOs: 1-13; or (e) a nucleotide sequence comprising one or more regions of about 100 bp or longer having at least 75% or greater sequence identity to any of SEQ ID NOs: 1-13.

In some embodiments, an AAV vector provided herein comprises a 5′ AAV inverted terminal repeat (ITR) and a 3′ AAV ITR flanking the heterologous nucleotide sequence, a promoter, and (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1.

In some embodiments, an AAV vector provided herein comprises, in 5′ to 3′ order: (i) the 5′ AAV ITR; (ii) the promoter; (iii) (a) the nucleotide sequence of any of SEQ ID NOs: 1-13; (b) a reverse nucleotide sequence of any of SEQ ID NOs: 1-13; (c) a complementary nucleotide sequence of any of SEQ ID NOs: 1-13; (d) a reverse complementary nucleotide sequence of any of SEQ ID NOs: 1-13; or (e) a nucleotide sequence comprising one or more regions of about 100 bp or longer having at least 75% or greater sequence identity to any of SEQ ID NOs: 1-13; (iv) the heterologous nucleotide sequence; and (v) the 3′ AAV ITR.

In some embodiments, an AAV vector provided herein comprises, in 5′ to 3′ order: (i) the 5′ AAV ITR; (ii) the promoter; (iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (iv) the heterologous nucleotide sequence; and (v) the 3′ AAV ITR.

In some embodiments, an AAV vector provided herein comprises, in 5′ to 3′ order: (i) the 5′ AAV ITR; (ii) (a) the nucleotide sequence of any of SEQ ID NOs: 1-13; (b) a reverse nucleotide sequence of any of SEQ ID NOs: 1-13; (c) a complementary nucleotide sequence of any of SEQ ID NOs: 1-13; (d) a reverse complementary nucleotide sequence of any of SEQ ID NOs: 1-13; or (e) a nucleotide sequence comprising one or more regions of about 100 bp or longer having at least 75% or greater sequence identity to any of SEQ ID NOs: 1-13; (iii) the promoter; (iv) the heterologous nucleotide sequence; and (v) the 3′ AAV ITR.

In some embodiments, an AAV vector provided herein comprises, in 5′ to 3′ order: (i) the 5′ AAV ITR; (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (iii) the promoter; (iv) the heterologous nucleotide sequence; and (v) the 3′ AAV ITR.

In some embodiments, an AAV vector provided herein comprises, in 5′ to 3′ order: (i) the 5′ AAV ITR; (ii) the promoter; (iii) the heterologous nucleotide sequence; (iv) (a) the nucleotide sequence of any of SEQ ID NOs: 1-13; (b) a reverse nucleotide sequence of any of SEQ ID NOs: 1-13; (c) a complementary nucleotide sequence of any of SEQ ID NOs: 1-13; (d) a reverse complementary nucleotide sequence of any of SEQ ID NOs: 1-13; or (e) a nucleotide sequence comprising one or more regions of about 100 bp or longer having at least 75% or greater sequence identity to any of SEQ ID NOs: 1-13; and (v) the 3′ AAV ITR.

In some embodiments, an AAV vector provided herein comprises, in 5′ to 3′ order: (i) the 5′ AAV ITR; (ii) the promoter; (iii) the heterologous nucleotide sequence; (iv) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (b) a reverse nucleotide sequence of any of SEQ ID NOs: 1-13; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 1; and (v) the 3′ AAV ITR.

The viral vectors and plasmids provided herein can be used in production of recombinant viral particles.

Provided herein is a viral particle (also referred to as a virion) comprising a vector disclosed herein. In some embodiments, the viral particle is a recombinant adenovirus particle. In some embodiments, the viral particle is a recombinant AAV (rAAV) particle. In some embodiments, the rAAV particle is an AAV-PHP.eB serotype particle. In some embodiments, the rAAV particle is an AAV9 serotype particle. In some embodiments, the rAAV particle is an AAV1, AAV2, AAV3, AAV4, AAV5, AAV6, AAV7, AAV8, AAV9, AAV10, AAV11, AAV12, AAV13, AAVrh.8, AAVrh.10, AAVrh32.33, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHP.eB, AAV-CAP.B10, AAV2-r3.45, AAV2-LSS, AAV2PFG, AAV2-PPS, AAV2-TLH or AAV2-GMN serotype particle, or any other AAV serotype particle now known or later discovered.

Provided herein is a population of viral particles comprising a plurality of viral particles disclosed herein. Provided herein is a population of recombinant adenovirus particles comprising a plurality of recombinant adenovirus particles disclosed herein. Provided herein is a population of rAAV particles comprising a plurality of rAAV particles disclosed herein.

In some embodiments, a vector provided herein is suitable for delivery via a non-viral delivery system. In some embodiments, a vector provided herein is formulated for delivery via a non-viral delivery system. In some embodiments, a non-viral delivery system is a lipid nanoparticle or an exosome. In some embodiments, non-viral systems for gene delivery may be lipid-based, polymer-based or other nanomaterial-based. Cationic lipids or cationic polymers can be complexed with nucleic acid molecules to produce synthetic vehicles for gene delivery.

Provided herein is a cell comprising any of the vectors or viral particles disclosed herein.

In some embodiments, the cell is a prokaryotic cell. In some embodiments, the cell is a eukaryotic cell. In some embodiments, the eukaryotic cell may be a mammalian cell, an insect cell, a plant cell, a fungal cell, a eukaryotic algae cell, a nematode cell, a protozoan cell, or a fish cell.

In some embodiments, the cell is a mammalian cell. In some embodiments, a mammalian cell is a Chinese hamster ovary (CHO) cell, a COS cell, a Vero cell, a SP2/0 cell, a NS/O myeloma cell, a human embryonic kidney cell, an immature hamster kidney cell, a HeLa cell, a human B cell, a cv-1/EBNA cell, an L cell, a 3T3 cell, a HEPG2 cell, a PerC6 cell, a 293 cell, or an MDCK cell. In some embodiments, a mammalian cell is a HEK293 cell. In some embodiments, a mammalian cell is a CHO cell.

In some embodiments, the cell is an insect cell. In some embodiments, an insect cell is a Spodoptera frugiperda cell (for example, the Sf9 or ExpiSf9™ cell lines). The Sf9 insect cell line (Thermo Fisher Scientific, Waltham, MA) is a clonal isolate derived from the parental S. frugiperda cell line IPLB-Sf-21-AE. ExpiSf9™ cells (Thermo Fisher Scientific, Waltham, MA) are a non-engineered derivative of Sf9 insect cells that have been adapted for high-density suspension growth.

### Pharmaceutical Compositions

Provided herein are pharmaceutical compositions comprising any of the vectors, viral particles, nucleic acid molecules, or populations of viral particles disclosed herein, and a pharmaceutically acceptable carrier, vehicle or diluent. “Pharmaceutically acceptable” refers to a material that is not toxic or otherwise undesirable, i.e., the material may be administered to a subject without causing any undesirable biological effects. In general, a pharmaceutically acceptable material has one or more benefits that outweigh any undesirable biological effect that the material may have. Undesirable biological effects may include, for example, excessive toxicity, irritation, allergic response, and other problems and complications.

For injection, the carrier will typically be a liquid. For other methods of administration, the carrier may be either solid or liquid.

In some embodiments, a pharmaceutical composition may comprise other medicinal agents, pharmaceutical agents, stabilizing agents, buffers, adjuvants and/or diluents.

In some embodiments, a pharmaceutical composition comprises at least one pharmaceutically acceptable carrier, excipient, and/or vehicle, for example, solvents, buffers, solutions, dispersion media, coatings, antibacterial agents, antifungal agents, isotonic agents, and absorption delaying agents. In some embodiments, the pharmaceutically acceptable carrier, excipient, and/or vehicle comprises saline, buffered saline, dextrose, water, glycerol, sterile isotonic aqueous buffer, or a combination thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like), or a suitable mixture thereof. In some embodiments, the compositions disclosed herein further comprise emulsifying or wetting agents, or pH buffering agents. Such species may be present in small amounts (e.g., less than 10% by weight of the composition, such as less than 5% by weight of the composition, 2% by weight of the composition, 1% by weight of the composition, or less).

In some embodiments, a pharmaceutical composition further comprises one or more other pharmaceutical ingredients, such as one or more preservatives or chemical stabilizers. Examples of preservatives and chemical stabilizers include, but are not limited to, chlorobutanol, potassium sorbate, sorbic acid, sulfur dioxide, propyl gallate, parabens, ethyl vanillin, glycerin, phenol, parachlorophenol, and albumin. In some embodiments, the compositions disclosed herein further comprise antibacterial agents and/or antifungal agents, such as parabens, chlorobutanol, phenol, sorbic acid, and thimerosal; isotonic agents, such as sugars and sodium chloride; and/or agents delaying absorption, such as aluminum monostearate and gelatin.

In some embodiments, a pharmaceutical composition is in a form of an injectable solution or dispersion, such as an aqueous solution or dispersion. In some embodiments, a pharmaceutical composition is a sterile powder for the extemporaneous preparation of sterile injectable solutions or dispersions. Dispersions may be prepared in water, glycerol, liquid polyethylene glycols, oils, or any combination thereof. Delivery vehicles such as liposomes, nanocapsules, microparticles, microspheres, lipid particles, vesicles, and the like, may be used for the introduction of the pharmaceutical compositions provided herein.

In some embodiments, a pharmaceutical composition comprises or consists of a sterile saline (e.g., pharmaceutical grade saline) solution and a vector. In some embodiments, a pharmaceutical composition comprises or consists of a vector and sterile water (e.g., pharmaceutical grade water). In some embodiments, a pharmaceutical composition comprises or consists of a vector and phosphate-buffered saline (PBS) (e.g., pharmaceutical grade PBS).

In some embodiments, a pharmaceutical composition provided herein comprises a vector and one or more excipients. In some embodiments, an excipient is water, a salt solution, an alcohol, a polyethylene glycol, gelatin, lactose, amylase, magnesium stearate, talc, silicic acid, viscous paraffin, hydroxymethylcellulose or polyvinylpyrrolidone.

In some embodiments, pharmaceutical compositions provided herein comprise a lipid moiety. For example, a vector may be introduced into preformed liposomes or lipoplexes made of mixtures of cationic lipids and neutral lipids. In some embodiments, vector complexes with mono- or poly-cationic lipids are formed without the presence of a neutral lipid. In some embodiments, a lipid moiety is selected to increase distribution of a pharmaceutical agent to a particular cell or tissue. In some embodiments, a lipid moiety is selected to increase distribution of a pharmaceutical agent to neurons.

In some embodiments, pharmaceutical compositions comprise a delivery system. Examples of delivery systems include, but are not limited to, liposomes and emulsions. Some delivery systems are useful for preparing certain pharmaceutical compositions including those comprising hydrophobic compounds. In some embodiments, organic solvents such as dimethylsulfoxide are used. In some embodiments, pharmaceutical compositions comprise a nanoparticle-based delivery system. In some embodiments, polylactic-co-glycolic acid (PLGA), poly (β-amino esters) (PBAE) and/or polyethylenimine (PEI) are used to formulate nanoparticles.

In some embodiments, pharmaceutical compositions comprise one or more tissue-specific delivery molecules designed to deliver a vector to specific tissues or cell types. For example, in some embodiments, pharmaceutical compositions include liposomes coated with a tissue-specific or a cell-specific antibody.

In some embodiments, pharmaceutical compositions comprise a cosolvent system. Some cosolvent systems comprise, for example, benzyl alcohol, a nonpolar surfactant, a water-miscible organic polymer, and an aqueous phase. In some embodiments, such cosolvent systems are used for hydrophobic compounds. A non-limiting example of such a cosolvent system is the VPD cosolvent system, which is a solution of absolute ethanol comprising 3% w/v benzyl alcohol, 8% w/v of the nonpolar surfactant Polysorbate 80 and 65% w/v polyethylene glycol 300. The proportions of such cosolvent systems may be varied considerably without significantly altering their solubility and toxicity characteristics. Furthermore, the identity of cosolvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

Provided herein is a nanoparticle comprising a vector disclosed herein. In some embodiments, a nanoparticle is a lipid nanoparticle. In some embodiments, a nanoparticle is a solid lipid nanoparticle (SLN).

In some embodiments, a pharmaceutical composition comprises a carrier and is formulated in aqueous solution, such as water or physiologically compatible buffers such as Hanks's solution, Ringer's solution, or physiological saline buffer. In some embodiments, other ingredients are included (e.g., ingredients that aid in solubility or serve as preservatives). In some embodiments, injectable suspensions are prepared using appropriate liquid carriers, suspending agents and the like. In some embodiments, pharmaceutical compositions for injection are prepared in unit dosage form, e.g., in ampoules or in multi-dose containers. Some pharmaceutical compositions for injection are suspensions, solutions or emulsions in oily or aqueous vehicles, and may contain formulatory agents such as suspending, stabilizing and/or dispersing agents. Exemplary solvents suitable for use in pharmaceutical compositions for injection include, but are not limited to, lipophilic solvents and fatty oils, such as sesame oil, synthetic fatty acid esters, such as ethyl oleate or triglycerides, and liposomes.

In some embodiments, vectors may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In some embodiments, a pharmaceutical composition is suitable or formulated for systemic administration. In some embodiments, a pharmaceutical composition is suitable or formulated for intravenous administration.

In some embodiments, a pharmaceutical composition is suitable or formulated for intracerebroventricular injection, intrathecal injection, intracarotid artery injection, or intraparenchymal injection.

Methods of Producing rAAV Particles

Provided herein are methods of producing rAAV particles by using any of the vectors and cells disclosed herein.

Provided herein is a method of producing a rAAV particle, the method comprising: (i) culturing a cell comprising a vector disclosed herein under conditions allowing for packaging the rAAV particle; and (ii) harvesting the cultured host cell or culture medium for collection of the rAAV particle.

In some embodiments, a method of producing a rAAV particle comprises providing to a cell: (a) a vector comprising two AAV ITRs located 5′ and 3′ to the polynucleotide sequences desired to be packaged into the rAAV particle, and (b) AAV sequences sufficient for replication of the nucleic acid template and encapsidation into AAV protein capsids (e.g., AAV rep sequences and AAV cap sequences encoding the AAV capsid subunits, also referred to as “helper functions”). Typically, the AAV rep and cap sequences will not be flanked by AAV ITRs, to prevent rescue and/or packaging of these sequences.

The vector (nucleic acid template), rep sequences, cap sequences, and any other helper functions required for producing the rAAV particles disclosed herein may be delivered to the packaging host cell using any appropriate genetic element. Further details on methods of preparing rAAV particles are provided in Sambrook et al., Molecular Cloning: A Laboratory Manual, Cold Spring Harbor Press, Cold Spring Harbor, NY; Fisher et al, J. Virol., 70:520-532 (1993) and U.S. Pat. No. 5,478,745.

The nucleic acid template and AAV rep and cap sequences are provided under conditions such that virus vector comprising the nucleic acid template packaged within the AAV protein capsid is produced in the cell. The method can further comprise the step of collecting the packaged virus vector from the cell. The packaged virus vector can be collected from the medium and/or by lysing the cells.

The cell can be a cell that is permissive for AAV viral replication. Any suitable cell known in the art may be employed. In some embodiments, the cell is a mammalian cell (e.g., a HEK293 cell). In some embodiments, the cell can be a trans-complementing packaging cell line that provides functions deleted from a replication-defective helper virus, e.g., HEK293 cells or other Ela trans-complementing cells. The helper sequences may be embedded in a chromosome or maintained as a stable extrachromosomal element.

In some embodiments, rAAV particles are produced using the triple transfection method, as described in U.S. Pat. No. 6,001,650. In some embodiments, the rAAVs are produced by transfecting a host cell with an AAV vector (i.e., AAV expression cassette) to be packaged into rAAV particles, an AAV helper function vector, and an accessory function vector. An AAV helper function vector encodes the “AAV helper function” sequences (i.e., rep and cap), which function in trans for productive AAV replication and encapsidation. Non-limiting examples of AAV helper function vectors include pHLP19 and pRep6cap6 vector, described in U.S. Pat. Nos. 6,001,650 and 6,156,303, respectively. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (e.g., other than herpes simplex virus type-1) and vaccinia virus.

In some embodiments, rAAVs are produced using recombinant baculovirus vectors. Production of rAAVs using baculovirus vectors is described in, for example, Urabe et al. (2002) Hum Gene Ther 13(16):1935-1943; Smith et al. (2009) Mol Ther 17(11):1888-1896; U.S. Pat. Nos. 8,945,918; 9,879,282; and US 2018/0371495. In some embodiments, a baculovirus vector genome is derived from Autographa californica multicapsid nucleopolyhedrovirus (AcMNPV), Bombyx mori nuclear polyhedrosis virus (BmNPV), Helicoverpa armigera (HearNPV) or Spodoptera exigua MNPV. Baculovirus vectors are used to produce recombinant AAVs in insect cells (e.g., Spodoptera frugiperda cells). In some embodiments, the Sf9 or ExpiSf9™ Spodoptera frugiperda cell lines are used to produce rAAVs. In some embodiments, methods of the disclosure comprise co-infecting insect cells with populations of recombinant baculoviruses (rBVs) to produce rAAV disclosed herein. At least two populations of rBVs may be used in the methods of the disclosure. Methods for generating recombinant baculovirus are known in the art (see, e.g., the Bac-to-Bac© Baculovirus Expression System (Thermo Fisher Scientific, Waltham, MA)).

In some embodiments, a rAAV particle produced by the methods provided herein comprises an AAV-PHP.eB capsid protein. In some embodiments, a rAAV particle produced by the methods provided herein comprises an AAV9 capsid protein. In some embodiments, a rAAV particle produced by the methods provided herein comprises an AAV1, AAV2, AAV3 (including types 3A and 3B), AAV4, AAV5, AAV6, AAV7, AAV8, AAV9, AAV10, AAV11, AAV12, AAV13, AAVrh32.33, AAVrh.8, AAVrh.10, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHP.eB, AAV-CAP.B10, AAV2-r3.45, AAV2-LSS, AAV2PFG, AAV2-PPS, AAV2-TLH or AAV2-GMN capsid protein, or any other AAV capsid protein now known or later discovered.

### Methods of Using Vectors

Provided herein is a method for selectively expressing the heterologous nucleotide sequence in neurons of the CNS in a subject in need thereof, the method comprising administering to the subject a vector, a viral particle, a population, a pharmaceutical composition, or a nanoparticle disclosed herein.

In some embodiments of the methods disclosed herein, the regulatory elements restrict the expression of the heterologous nucleotide sequence to neurons of the CNS with at least about 75%, about 80%, about 85%, about 90%, about 95%, or about 99% specificity for this cell type. In some embodiments of the methods disclosed herein, the regulatory elements restrict the expression of the heterologous nucleotide sequence to neurons of the CNS with at least about 75%, about 80%, about 85%, about 90%, about 95%, or about 99% selectivity for this cell type.

In some embodiments of the methods disclosed herein, the regulatory elements express the heterologous nucleotide sequence in non-neuronal cells of the CNS (e.g., glial cells or endothelial cells) with at most about 25%, about 20%, about 15%, about 10%, about 5%, or about 1% specificity for this cell type. In some embodiments of the methods disclosed herein, the regulatory elements express the heterologous nucleotide sequence in non-neuronal cells of the CNS (e.g., glial cells or endothelial cells) with at most about 25%, about 20%, about 15%, about 10%, about 5%, or about 1% selectivity for this cell type.

In some embodiments of the methods disclosed herein, the heterologous nucleotide sequence expression is enriched in specific regions of the CNS. The terms “enrichment” and “sensitivity” can be used interchangeably. The regulatory elements provided herein, when integrated in a DNA-based expression vector, are capable of driving functional expression of the heterologous nucleotide sequence to specific regions of the brain with above 10% sensitivity for neurons in a given target region (i.e., the forebrain) while preventing the expression of the heterologous nucleotide sequence (e.g., less than 5% sensitivity for cells or less than 2% sensitivity for cells) in other regions of the brain.

In some embodiments of the methods disclosed herein, the regulatory elements restrict the expression of the heterologous nucleotide sequence to neurons of the CNS with at least about 70% specificity for this cell type; and enrich the expression of the heterologous nucleotide sequence in specific regions of the brain with above 10% sensitivity for neurons in a given target region.

In some embodiments of the methods disclosed herein, the regulatory elements restrict the expression of the heterologous nucleotide sequence to neurons of the CNS with at least about 80% specificity for this cell type; and enrich the expression of the heterologous nucleotide sequence in specific regions of the brain with above 10% sensitivity for neurons in a given target region.

In some embodiments of the methods disclosed herein, the regulatory elements restrict the expression of the heterologous nucleotide sequence to neurons of the CNS with at least about 90% specificity for this cell type; and enrich the expression of the heterologous nucleotide sequence in specific regions of the brain with above 10% sensitivity for neurons in a given target region.

In some embodiments of the methods disclosed herein, the heterologous nucleotide sequence expression is enriched in neurons of one or more of: the forebrain, the cerebellum, and the spinal cord. In some embodiments, the heterologous nucleotide sequence expression is enriched in neurons of the forebrain and the cerebellum. In some embodiments, the heterologous nucleotide sequence expression is enriched in neurons of the forebrain. In some embodiments, the heterologous nucleotide sequence expression is enriched in neurons of the cerebellum. In some embodiments, the heterologous nucleotide sequence expression is enriched in neurons of the spinal cord. In some embodiments, the heterologous nucleotide sequence expression is enriched in neurons of neurogenic niches.

In some embodiments, the vector comprises the nucleotide sequence of SEQ ID NO:1 or SEQ ID NO: 10, and the heterologous nucleotide sequence expression is enriched in neurons of the CNS (e.g., pan-neuronal expression across all brain regions).

In some embodiments, the vector comprises the nucleotide sequence of SEQ ID NO: 2 or SEQ ID NO: 9, and the heterologous nucleotide sequence expression is enriched in neurons of the forebrain and the cerebellum.

In some embodiments, the vector comprises the nucleotide sequence of SEQ ID NO: 4, SEQ ID NO: 5, or SEQ ID NO: 11 and the heterologous nucleotide sequence expression is enriched in neurons of the forebrain.

In some embodiments, the vector comprises the nucleotide sequence of SEQ ID NO: 6, and the heterologous nucleotide sequence expression is enriched in neurons of the cerebellum and hippocampus.

In some embodiments, the vector, the viral particle, the population, the pharmaceutical composition, or the nanoparticle is administered to the subject via systemic administration. In some embodiments, the vector, the viral particle, the population, the pharmaceutical composition, or the nanoparticle is administered to the subject intravenously. In some embodiments, the vector, the viral particle, the population, the pharmaceutical composition, or the nanoparticle is administered to the subject via injection into the CNS. In some embodiments, the vector, the viral particle, the population, the pharmaceutical composition, or the nanoparticle is administered to the subject via ICV injection.

In some embodiments, the subject has or is suspected of having a disease or disorder associated with inappropriate expression of a protein or a nucleic acid in neurons of the CNS. In some embodiments, the subject has or is suspected of having a disease or disorder associated with inappropriate expression of a protein or a nucleic acid in neurons of one or more of: the forebrain, the cerebellum, and the spinal cord. In some embodiments, the subject has or is suspected of having a disease or disorder associated with inappropriate expression of a protein or a nucleic acid in neurons of neurogenic niches.

In some embodiments, the subject has or is suspected of having a neurodegenerative, neurological, neurodevelopmental, or neuropsychiatric disease or disorder.

In some embodiments, administering to the subject a vector, a viral particle, a population, a pharmaceutical composition, or a nanoparticle disclosed herein is used to treat or prevent a disease or disorder associated with inappropriate expression of a protein or a nucleic acid in neurons of the CNS. In some embodiments, administering to the subject a vector, a viral particle, a population, a pharmaceutical composition, or a nanoparticle disclosed herein is used to reduce or ameliorate one or more symptoms of a disease or disorder associated with inappropriate expression of a protein or a nucleic acid in neurons of the CNS.

In some embodiments, the subject is human. In some embodiments, the subject is less than 2 years old. In some embodiments, the subject is between about 2 years old and about 18 years old. In some embodiments, the subject is older than 18 years.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the terms “specificity” and “sensitivity” are defined as follows. For quantification of colocalization analysis, cells expressing the indicated reporter are counted using only the corresponding color channel, and cells expressing the pan-neuronal marker NeuN (i.e., neurons) are identified independently using only the corresponding color channel. The ratio of cells co-expressing both markers over the total number of cells expressing only the reporter is then calculated and reported as mean±s.e.m (standard error of the mean) (i.e., specificity). The ratio of cells co-expressing both markers over the total number of cells expressing the pan-neuronal marker is calculated and reported as mean±s.e.m (i.e., sensitivity).

As used herein, the term “vector” refers to a delivery vehicle for a polynucleotide (e.g., a heterologous nucleotide sequence). A vector may be used to deliver the heterologous nucleotide sequence to a host cell. In some embodiments, the vector comprises a heterologous nucleotide sequence encoding a protein or a gene silencing nucleic acid operatively inserted therein and enables the expression of this protein or gene silencing nucleic acid in a genetic engineering recombinant technique. The vector can be used to transform, transduce or transfect a host cell and to enable the genetic material element carried by the vector to be expressed in the host cell. The “vector” may be any suitable vector, including chromosomal, non-chromosomal, and synthetic nucleic acid vectors.

As used herein, the term “heterologous” refers to a substance coming from some source other than its native source. For example, the term “heterologous nucleotide sequence” refers to a nucleotide sequence from a non-native source or location that has been artificially supplied to a biological system.

As used herein, the term “gRNA molecule” or “gRNA” refers to a guide RNA that is capable of targeting a CRISPR nuclease or a nuclease-deficient CRISPR-associated protein to a target nucleic acid. Depending on context, the term “gRNA molecule” refers to a guide ribonucleic acid or to a nucleic acid encoding a gRNA.

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular Systems Biology 7:539. For the purposes of calculating identity to the sequence, extensions, such as tags, are not included.

As used herein, a regulatory sequence (e.g., a promoter or a genetic regulatory element provided herein) is considered to be “operatively linked” when it is in a functional location and orientation in relation to a nucleic acid sequence it regulates to control transcriptional initiation and/or expression of that sequence.

As used herein, the term “self-complementary” when referring to an AAV vector refers to an AAV vector comprising a nucleic acid (i.e., a DNA) that forms a dimeric inverted repeat molecule that spontaneously anneals, resulting in earlier and more robust transgene expression compared with conventional single-strand (ss) AAV genomes. See, e.g., McCarty, Molecular Therapy 16(10):1648-1656 (2008). Unlike conventional ssAAV, self-complementary AAV (scAAV) can bypass second-strand synthesis, the rate-limiting step for gene expression. Moreover, double-stranded scAAV is less prone to DNA degradation after viral transduction, thereby increasing the number of copies of stable episomes.

As used herein, a “therapeutically effective amount” is the amount of a vector, a viral particle, a population of viral particles, a pharmaceutical composition, or a nanoparticle provided herein that is effective to treat or prevent a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

In the present description, any concentration range, percentage range, ratio range, or integer range is to be understood to include the value of any integer within the recited range and, when appropriate, fractions thereof (such as one tenth and one hundredth of an integer), unless otherwise indicated.

The use of the alternative (e.g., “or”) should be understood to mean either one, both, or any combination thereof of the alternatives.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

The section headings used herein are for organizational purposes only and are not to be construed as limiting the subject matter described.

### NUMBERED EMBODIMENTS

Notwithstanding the appended claims, the disclosure sets forth the following numbered embodiments:

Embodiment 1. A vector comprising (i) a heterologous nucleotide sequence; and (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 base pairs (bp) or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

Embodiment 2. The vector of embodiment 1, wherein the vector further comprises a promoter.

Embodiment 3. The vector of embodiment 2, comprising, in 5′ to 3′ order: (i) the promoter; (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (iii) the heterologous nucleotide sequence.

Embodiment 4. The vector of embodiment 2, comprising, in 5′ to 3′ order: (i)
(a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (ii) the promoter; and (iii) the heterologous nucleotide sequence.

Embodiment 5. The vector of embodiment 2, comprising, in 5′ to 3′ order: (i) the promoter; (ii) the heterologous nucleotide sequence; and (iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

Embodiment 6. The vector of any one of embodiments 1-5, wherein the vector is a non-viral vector.

Embodiment 7. The vector of embodiment 6, wherein the non-viral vector is a plasmid vector.

Embodiment 8. The vector of any one of embodiments 1-5, wherein the vector is a viral vector.

Embodiment 9. The vector of embodiment 8, wherein the viral vector is an adeno-associated virus (AAV) vector or an adenovirus vector.

Embodiment 10. The vector of embodiment 9, wherein the AAV vector comprises a 5′ AAV inverted terminal repeat (ITR) and a 3′ AAV ITR flanking the heterologous nucleotide sequence, a promoter, and (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

Embodiment 11. The vector of embodiment 10, comprising, in 5′ to 3′ order: (i) the 5′ AAV ITR; (ii) the promoter; (iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (iv) the heterologous nucleotide sequence; and (v) the 3′ AAV ITR.

Embodiment 12. The vector of embodiment 10, comprising, in 5′ to 3′ order: (i) the 5′ AAV ITR; (ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (iii) the promoter; (iv) the heterologous nucleotide sequence; and (v) the 3′ AAV ITR.

Embodiment 13. The vector of embodiment 10, comprising, in 5′ to 3′ order: (i) the 5′ AAV ITR; (ii) the promoter; (iii) the heterologous nucleotide sequence; (iv) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; (d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or (e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and (v) the 3′ AAV ITR.

Embodiment 14. The vector of any one of embodiments 2-13, wherein the promoter comprises an RNA-polymerase II binding site, a TATA box, a TF2B recognition element (BRE), a motif 10 element (MTE), or a downstream promoter element (DPE).

Embodiment 15. The vector of any one of embodiments 2-13, wherein the promoter is a minimal promoter.

Embodiment 16. The vector of embodiment 15, wherein the minimal promoter is a human beta-globin minimal promoter.

Embodiment 17. The vector of any one of embodiments 1-16, wherein the vector further comprises an enhancer.

Embodiment 18. The vector of any one of embodiments 1-17, wherein the vector further comprises or encodes a polyadenylation signal sequence.

Embodiment 19. The vector of any one of embodiments 1-18, wherein the vector further comprises or encodes a Kozak sequence.

Embodiment 20. The vector of any one of embodiments 1-19, wherein the vector further comprises or encodes a woodchuck hepatitis virus post-transcriptional element (WPRE), a hepatitis B virus posttranscriptional regulatory element, an RNA transport element (RTE), a WPRE3, or a wsl3 regulatory element.

Embodiment 21. The vector of any one of embodiments 1-20, wherein the vector further comprises an artificial intron.

Embodiment 22. The vector of any one of embodiments 1-21, wherein the vector further encodes a 2A self-cleaving peptide.

Embodiment 23. The vector of any one of embodiments 1-22, wherein the vector further comprises or encodes an internal ribosome entry site (IRES).

Embodiment 24. The vector of any one of embodiments 1-23, wherein the heterologous nucleotide sequence encodes a protein.

Embodiment 25. The vector of any one of embodiments 1-23, wherein the heterologous nucleotide sequence encodes a gene silencing nucleic acid.

Embodiment 26. The vector of any one of embodiments 1-23, wherein the heterologous nucleotide sequence encodes a CRISPR-associated protein.

Embodiment 27. The vector of embodiment 26, wherein the heterologous nucleotide sequence further encodes a guide RNA (gRNA).

Embodiment 28. A viral particle comprising the vector of any one of embodiments 1-5 and 8-27.

Embodiment 29. The viral particle of embodiment 28, wherein the viral particle is a recombinant adenovirus particle.

Embodiment 30. The viral particle of embodiment 28, wherein the viral particle is a recombinant AAV (rAAV) particle.

Embodiment 31. The viral particle of embodiment 30, wherein the rAAV particle is an AAV1, AAV2, AAV3, AAV4, AAV5, AAV6, AAV7, AAV8, AAV9, AAV10, AAV11, AAV12, AAV13, AAVrh.8, AAVrh.10, AAVrh32.33, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHP.eB, AAV-CAP.B10, AAV2-r3.45, AAV2-LSS, AAV2PFG, AAV2-PPS, AAV2-TLH or AAV2-GMN serotype particle.

Embodiment 32. A population of viral particles comprising a plurality of viral particles of any one of embodiments 28-32.

Embodiment 33. A pharmaceutical composition comprising the vector of any one of embodiments 1-27, the viral particle of any one of embodiments 28-32 or the population of embodiment 32, and a pharmaceutically acceptable carrier, vehicle or diluent.

Embodiment 34. A nanoparticle comprising the vector of any one of embodiments 1-7 and 14-27.

Embodiment 35. A cell comprising the vector of any one of embodiments 1-27 or the viral particle of any one of embodiments 28-32.

Embodiment 36. The cell of embodiment 35, wherein the cell is a mammalian cell or an insect cell.

Embodiment 37. A method of producing a rAAV particle, the method comprising: (i) culturing the cell of embodiment 35 or 36 under conditions allowing for packaging the rAAV particle; and (ii) harvesting the cultured host cell or culture medium for collection of the rAAV particle.

Embodiment 38. A method for selectively expressing the heterologous nucleotide sequence in neurons of the central nervous system (CNS) in a subject in need thereof, the method comprising administering to the subject the vector of any one of embodiments 1-27, the viral particle of any one of embodiments 28-32, the population of embodiment 32, the pharmaceutical composition of embodiment 33, or the nanoparticle of embodiment 34.

Embodiment 39. The method of embodiment 38, wherein the heterologous nucleotide sequence expression is enriched in specific regions of the CNS.

Embodiment 40. The method of embodiment 38, wherein the heterologous nucleotide sequence expression is enriched in neurons of one or more of: the forebrain, the cerebellum, and the spinal cord.

Embodiment 41. The method of embodiment 38, wherein the heterologous nucleotide sequence expression is enriched in neurons of the forebrain and the cerebellum.

Embodiment 42. The method of embodiment 38, wherein the heterologous nucleotide sequence expression is enriched in neurons of the forebrain.

Embodiment 43. The method of embodiment 38, wherein the heterologous nucleotide sequence expression is enriched in neurons of the cerebellum.

Embodiment 44. The method of embodiment 38, wherein the heterologous nucleotide sequence expression is enriched in neurons of the spinal cord.

Embodiment 45. The method of embodiment 38, wherein the vector comprises the nucleotide sequence of SEQ ID NO:1 or SEQ ID NO: 10, and wherein the heterologous nucleotide sequence expression is enriched in neurons of the CNS.

Embodiment 46. The method of embodiment 38, wherein the vector comprises the nucleotide sequence of SEQ ID NO: 2, or SEQ ID NO: 9, and wherein the heterologous nucleotide sequence expression is enriched in neurons of the forebrain and the cerebellum.

Embodiment 47. The method of embodiment 38, wherein the vector comprises the nucleotide sequence of SEQ ID NO: 4, SEQ ID NO: 5, or SEQ ID NO: 11, and wherein the heterologous nucleotide sequence expression is enriched in neurons of the forebrain.

Embodiment 48. The method of embodiment 38, wherein the vector comprises the nucleotide sequence of SEQ ID NO: 6, and wherein the heterologous nucleotide sequence expression is enriched in neurons of the cerebellum and hippocampus.

Embodiment 49. The method of any one of embodiments 38-48, wherein the vector, the viral particle, the population, the pharmaceutical composition, or the nanoparticle is administered to the subject intravenously.

Embodiment 50. The method of any one of embodiments 38-48, wherein the vector, the viral particle, the population, the pharmaceutical composition, or the nanoparticle is administered to the subject via injection into the CNS.

The disclosure will be further clarified by the following example, which is intended to be purely exemplary of the disclosure and in no way limiting.

### EXAMPLE

### Generation and Analysis of Regulatory Elements

### Identification of Regulatory Elements

The sequences of the 13 synthetic regulatory elements were engineered using a proprietary method. The Regulatory Element High Throughput Screening (RE-HTS) platform combines a comprehensive analysis of genomic and epigenomic next-generation sequencing data relevant to the target cell-type to identify short DNA sequences predicted to be capable of restricting viral transgene expression to the target cell type.

rAAV Cloning and Production

The regulatory elements were then synthesized de novo and individually cloned into the custom vector. The custom vector contained (in 5′ to 3′ order) a 5′ AAV ITR, a cloning site, the coding sequence of the red-fluorescent reporter tDTomato or the coding sequence of dCas9, a WPRE, a poly-adenylation sequence and a 3′ AAV ITR. This vector was synthesized de novo and used as the common backbone for all the constructs. A “dCas” or “dead Cas” protein is a nuclease-deficient CRISPR-associated protein (also known as a catalytically inactive CRISPR nuclease). The constructs comprised one of the regulatory elements immediately followed by a minimal promoter sequence, immediately downstream of the 5′ AAV ITR. Full plasmid Sanger sequencing was then performed to ensure the integrity of each construct. The corresponding rAAVs were produced in-house using standard production methods. PEI was used for transfection and OptiPrep gradient (Sigma, USA) was used for viral particle purification. All batches produced were in the range of 10E+11 to 10E+13 viral genomes per ml.

### Systemic Viral Injections

For systemic injection in adult mice, 10E+11 viral particles produced with the AAV capsid PHP.eB (Chan et al., Nat. Neurosci. 20, 1172-1179 (2017)) were injected in the tail vein of the animal. Post-intervention monitoring was performed for five days following the injection.

### Immunohistochemistry

All animals injected with the virus were trans-cardially perfused with 4% paraformaldehyde (PFA). The organs were placed in 4% PFA for 3 h then sectioned using a Leica VTS1200 vibrosector. Floating sections were permeabilized with 0.3% Triton X 100, 5% normal donkey serum, and PBS for 30 minutes. The sections were then incubated overnight in 0.1% Triton X 100 with the indicated combination of the following primary antibodies at 4° C.: rabbit anti-Ds-red at 1:1000 (Clontech, 632496) and mouse anti-NeuN at 1:500 (Invitrogen, MA5-33103). The sections were then washed three times with PBS, incubated with Alexa Fluor-conjugated secondary antibodies at 1:1,000 (Invitrogen, USA), counterstained with DAPI (Sigma, USA) and mounted on glass slides using Fluoromount-G (Sigma, USA).

### Quantifications, Statistics and Reproducibility

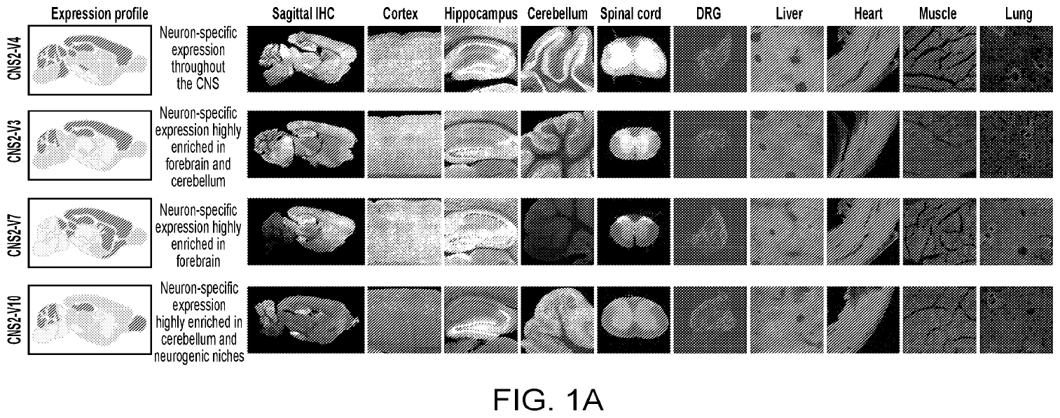

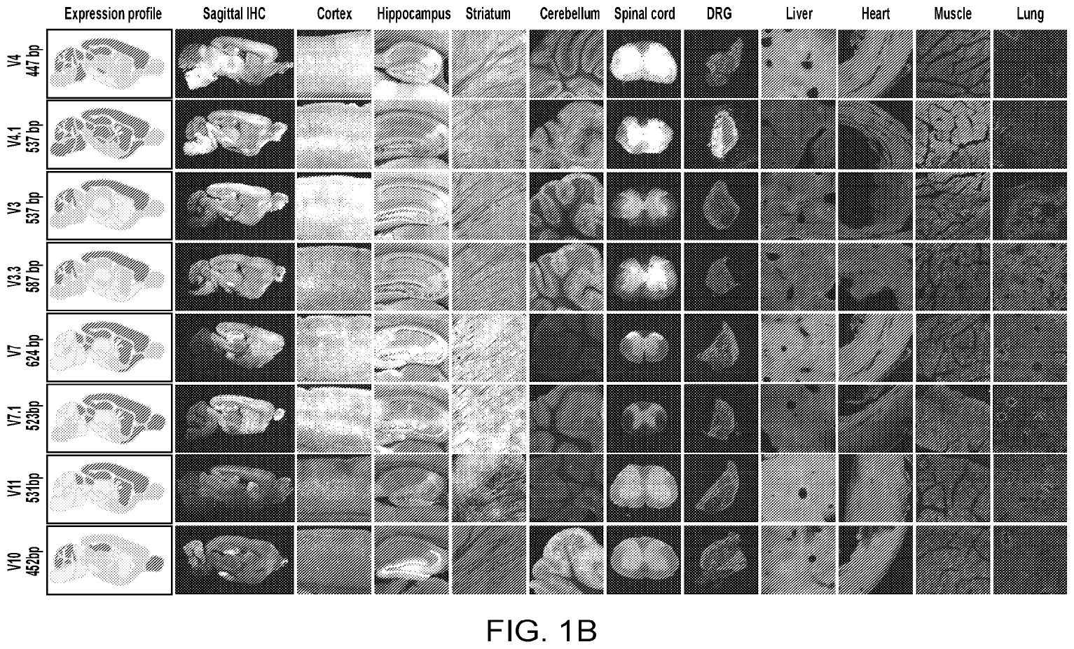

For expression analysis, fluorescence images were taken at a standardized magnification and exposure time for each organ. Representative images that are comparable across constructs are presented in FIG. 1A and FIG. 1B. Brain heatmap and representative images of transgene expression driven by the indicated engineered regulatory element across indicated brain regions and organs are shown. Note that the heatmap has 5 levels, with darker shades corresponding to a higher percentage of cells expressing the transgene (i.e., darkest tone is the strongest expression observed, and lightest tone corresponds to little to no expression).

Expression profiles of the regulatory elements CNS2_V4 (SEQ ID NO: 1), CNS2_V3 (SEQ ID NO: 2), CNS2_V7 (SEQ ID NO: 4), and CNS2_V10 (SEQ ID NO: 6) are shown in FIG. 1A. CNS2_V4 exhibited neuron-specific expression throughout the CNS. CNS2_V3 exhibited neuron-specific expression highly enriched in forebrain and cerebellum. CNS2_V7 exhibited neuron-specific expression highly enriched in forebrain. CNS2_V10 exhibited neuron-specific expression highly enriched in cerebellum and neurogenic niches.

Expression profiles of the regulatory elements CNS2_V4 (SEQ ID NO: 1), CNS2_V4.1 (SEQ ID NO: 10); CNS2_V3 (SEQ ID NO: 2), CNS2_V3.3 (SEQ ID NO: 9); CNS2_V7 (SEQ ID NO: 4), CNS2_V7.1 (SEQ ID NO: 11), CNS2_V11 (SEQ ID NO: 5), and CNS2_V10 (SEQ ID NO: 6) are shown in FIG. 1B. CNS2_V4 and CNS2_V4.1 exhibited pan-neuronal expression across all brain regions. CNS2_V3 and CNS2_V3.3 exhibited forebrain and cerebellum enriched expression. CNS2_V7, CNS2_V7.1, and CNS2_V11 exhibited forebrain enriched expression. CNS2_V10 exhibited cerebellum and hippocampus enriched expression.

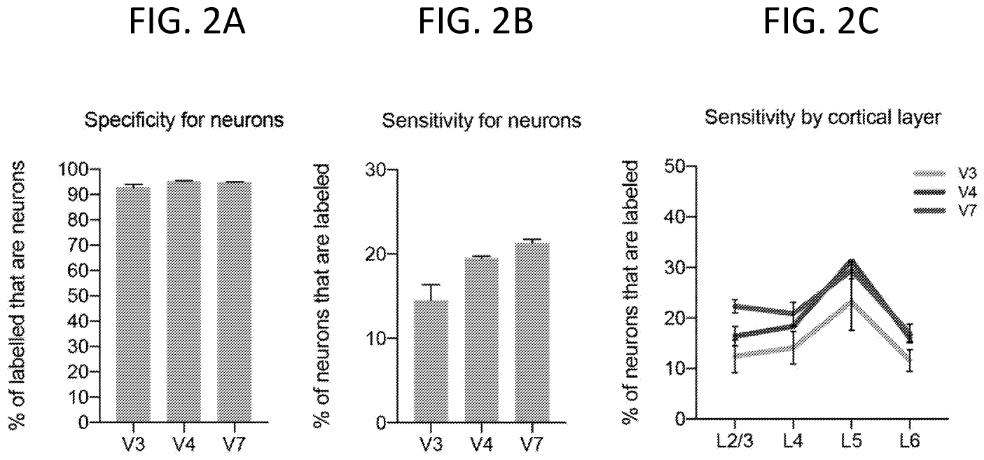

For quantification of colocalization analysis, cells expressing the indicated reporter were counted using only the corresponding color channel, and cells expressing the pan-neuronal marker NeuN (i.e., neurons) were identified independently using only the corresponding color channel. The ratio of cells co-expressing both markers over the total number of cells expressing only the reporter was then calculated and reported as mean±s.e.m (i.e., specificity). The ratio of cells co-expressing both markers over the total number of cells expressing the pan-neuronal marker was also calculated and reported as mean±s.e.m (i.e., sensitivity). Quantifications were performed using a minimum of two independent biological replicates and all replicates were highly consistent. Results corresponding to the data in FIG. 1A are shown in FIG. 2A, FIG. 2B, and FIG. 2C.

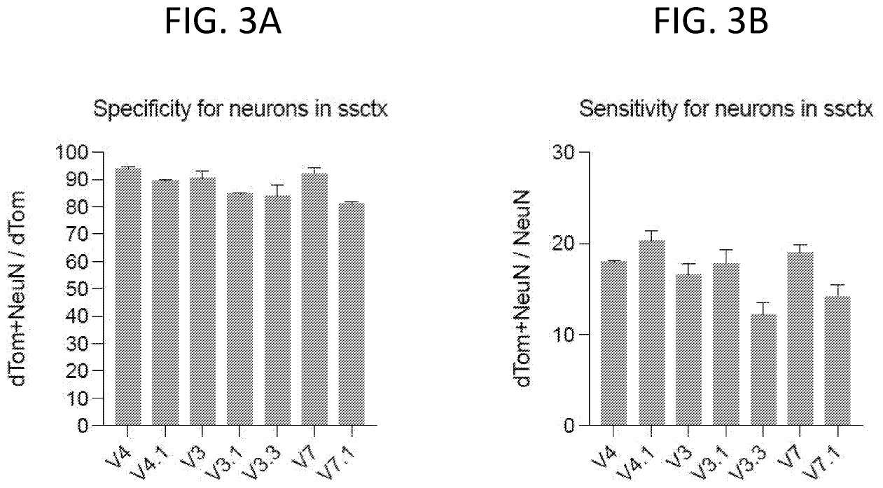

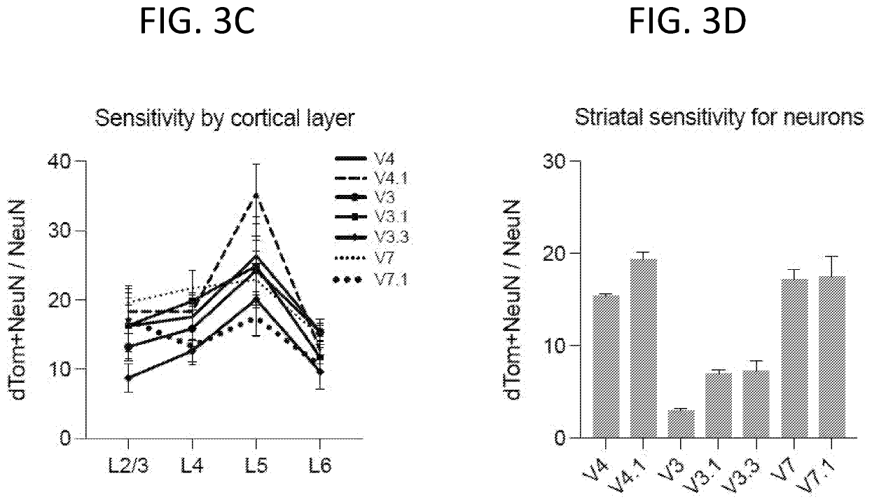

Results corresponding to the data in FIG. 1B are shown in FIG. 3A-FIG. 3D. Results of quantitative analysis of intensity, specificity and sensitivity of transgene expression following adult mouse systemic injection using the PHP.eB capsid and 3 weeks of incubation are shown. FIG. 3A shows percent specificity of transgene expression (dTomato) for neurons (determined by NeuN expression) within the somatosensory cortex. FIG. 3B shows percent sensitivity (percentage of the neuronal population captured) of transgene expression for neurons within the somatosensory cortex following systemic administration. FIG. 3C shows percent sensitivity of transgene expression across cortical layers within the somatosensory cortex. FIG. 3D shows percent sensitivity of transgene expression for neurons within the striatum.

Upon systemic delivery of the rAAV (i.e., across the body by injection in the circulatory system), these sequences did not elicit transgene expression in any organ other than the brain.

### Intracerebroventricular Viral Injections

For ICV injection in postnatal mice, 5E+10 viral particles produced with the AAV capsid AAV9 were injected at PND1. Animals were collected at postnatal day 29 (PND)29, after 4 weeks of incubation.

Analysis of Early Postnatal ICV Injection with Regulatory Element Driving Either dTomato or dCas9 in AAV9

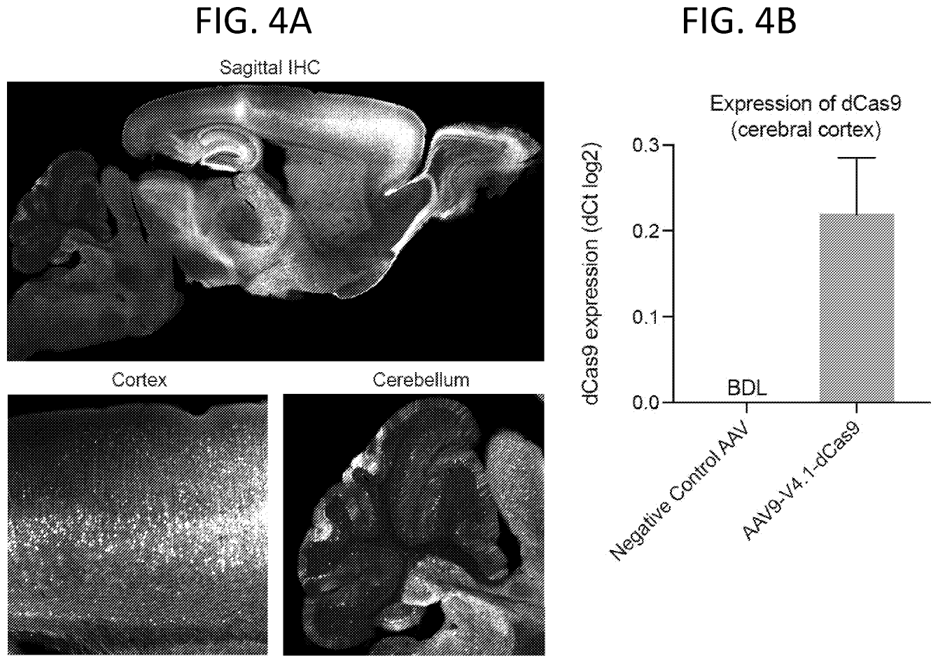

Representative images of transgene expression following ICV injection at P1 using the AAV9 capsid and 4 weeks of incubation (animals collected at P29) are shown in FIG. 4A and FIG. 4B. FIG. 4A shows images of fluorescent reporter (dTomato) expression driven by CNS2_V4.1 (SEQ ID NO: 10) within the brain. FIG. 4B shows analysis of expression of dCas9 transgene expression driven by CNS2_V4.1 (SEQ ID NO: 10) detected by qPCR.

The data suggest that the regulatory elements retain their properties across method of injection, age of injection, transgene and serotype. The CNS2_V4.1 regulatory element showed the same expression profile upon ICV injection with the AAV9 capsid at PND1 compared to systemic injection with the AAV PHP.eB capsid in adults.

What is claimed is:

1. A vector comprising
(i) a heterologous nucleotide sequence; and
(ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 base pairs (bp) or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

2. The vector of claim 1, wherein the vector further comprises a promoter.

3. The vector of claim 2, comprising, in 5′ to 3′ order:
(i) the promoter;
(ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and
(iii) the heterologous nucleotide sequence.

4. The vector of claim 2, comprising, in 5′ to 3′ order:
(i) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and
(ii) the promoter; and
(iii) the heterologous nucleotide sequence.

5. The vector of claim 2, comprising, in 5′ to 3′ order:
(i) the promoter;
(ii) the heterologous nucleotide sequence; and
(iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

6. The vector of any one of claims 1-5, wherein the vector is a non-viral vector.

7. The vector of claim 6, wherein the non-viral vector is a plasmid vector.

8. The vector of any one of claims 1-5, wherein the vector is a viral vector.

9. The vector of claim 8, wherein the viral vector is an adeno-associated virus (AAV) vector or an adenovirus vector.

10. The vector of claim 9, wherein the AAV vector comprises a 5′ AAV inverted terminal repeat (ITR) and a 3′ AAV ITR flanking the heterologous nucleotide sequence, a promoter, and
(a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11.

11. The vector of claim 10, comprising, in 5′ to 3′ order:
(i) the 5′ AAV ITR;
(ii) the promoter;
(iii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(iv) the heterologous nucleotide sequence; and
(v) the 3′ AAV ITR.

12. The vector of claim 10, comprising, in 5′ to 3′ order:
(i) the 5′ AAV ITR;
(ii) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(iii) the promoter;
(iv) the heterologous nucleotide sequence; and
(v) the 3′ AAV ITR.

13. The vector of claim 10, comprising, in 5′ to 3′ order:
(i) the 5′ AAV ITR;
(ii) the promoter;
(iii) the heterologous nucleotide sequence;
(iv) (a) the nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(b) a reverse nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(c) a complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11;
(d) a reverse complementary nucleotide sequence of any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; or
(e) a nucleotide sequence comprising one or more regions of about 100 bp or longer having at least 75% or greater sequence identity to any of SEQ ID NO: 1, SEQ ID NO: 2, SEQ ID NO: 4, SEQ ID NO: 5, SEQ ID NO: 6, SEQ ID NO: 9, SEQ ID NO: 10, and SEQ ID NO: 11; and
(v) the 3′ AAV ITR.

14. The vector of any one of claims 2-13, wherein the promoter comprises an RNA-polymerase II binding site, a TATA box, a TF2B recognition element (BRE), a motif 10 element (MTE), or a downstream promoter element (DPE).

15. The vector of any one of claims 2-13, wherein the promoter is a minimal promoter.

16. The vector of claim 15, wherein the minimal promoter is a human beta-globin minimal promoter.

17. The vector of any one of claims 1-16, wherein the vector further comprises an enhancer.

18. The vector of any one of claims 1-17, wherein the vector further comprises or encodes a polyadenylation signal sequence.

19. The vector of any one of claims 1-18, wherein the vector further comprises or encodes a Kozak sequence.

20. The vector of any one of claims 1-19, wherein the vector further comprises or encodes a woodchuck hepatitis virus post-transcriptional element (WPRE), a hepatitis B virus posttranscriptional regulatory element, an RNA transport element (RTE), a WPRE3, or a wsl3 regulatory element.

21. The vector of any one of claims 1-20, wherein the vector further comprises an artificial intron.

22. The vector of any one of claims 1-21, wherein the vector further encodes a 2A self-cleaving peptide.

23. The vector of any one of claims 1-22, wherein the vector further comprises or encodes an internal ribosome entry site (IRES).

24. The vector of any one of claims 1-23, wherein the heterologous nucleotide sequence encodes a protein.

25. The vector of any one of claims 1-23, wherein the heterologous nucleotide sequence encodes a gene silencing nucleic acid.

26. The vector of any one of claims 1-23, wherein the heterologous nucleotide sequence encodes a CRISPR-associated protein.

27. The vector of claim 26, wherein the heterologous nucleotide sequence further encodes a guide RNA (gRNA).

28. A viral particle comprising the vector of any one of claims 1-5 and 8-27.

29. The viral particle of claim 28, wherein the viral particle is a recombinant adenovirus particle.

30. The viral particle of claim 28, wherein the viral particle is a recombinant AAV (rAAV) particle.

31. The viral particle of claim 30, wherein the rAAV particle is an AAV1, AAV2, AAV3, AAV4, AAV5, AAV6, AAV7, AAV8, AAV9, AAV10, AAV11, AAV12, AAV13, AAVrh.8, AAVrh.10, AAVrh32.33, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHP.eB, AAV-CAP.B10, AAV2-r3.45, AAV2-LSS, AAV2PFG, AAV2-PPS, AAV2-TLH or AAV2-GMN serotype particle.

32. A population of viral particles comprising a plurality of viral particles of any one of claims 28-32.

33. A pharmaceutical composition comprising the vector of any one of claims 1-27, the viral particle of any one of claims 28-32 or the population of claim 32, and a pharmaceutically acceptable carrier, vehicle or diluent.

34. A nanoparticle comprising the vector of any one of claims 1-7 and 14-27.

35. A cell comprising the vector of any one of claims 1-27 or the viral particle of any one of claims 28-32.

36. The cell of claim 35, wherein the cell is a mammalian cell or an insect cell.

37. A method of producing a rAAV particle, the method comprising:
(i) culturing the cell of claim 35 or 36 under conditions allowing for packaging the rAAV particle; and
(ii) harvesting the cultured host cell or culture medium for collection of the rAAV particle.

38. A method for selectively expressing the heterologous nucleotide sequence in neurons of the central nervous system (CNS) in a subject in need thereof, the method comprising administering to the subject the vector of any one of claims 1-27, the viral particle of any one of claims 28-32, the population of claim 32, the pharmaceutical composition of claim 33, or the nanoparticle of claim 34.

39. The method of claim 38, wherein the heterologous nucleotide sequence expression is enriched in specific regions of the CNS.

40. The method of claim 38, wherein the heterologous nucleotide sequence expression is enriched in neurons of one or more of: the forebrain, the cerebellum, and the spinal cord.

41. The method of claim 38, wherein the heterologous nucleotide sequence expression is enriched in neurons of the forebrain and the cerebellum.

42. The method of claim 38, wherein the heterologous nucleotide sequence expression is enriched in neurons of the forebrain.

43. The method of claim 38, wherein the heterologous nucleotide sequence expression is enriched in neurons of the cerebellum.

44. The method of claim 38, wherein the heterologous nucleotide sequence expression is enriched in neurons of the spinal cord.

45. The method of claim 38, wherein the vector comprises the nucleotide sequence of SEQ ID NO:1 or SEQ ID NO: 10, and wherein the heterologous nucleotide sequence expression is enriched in neurons of the CNS.

46. The method of claim 38, wherein the vector comprises the nucleotide sequence of SEQ ID NO: 2 or SEQ ID NO: 9, and wherein the heterologous nucleotide sequence expression is enriched in neurons of the forebrain and the cerebellum.

47. The method of claim 38, wherein the vector comprises the nucleotide sequence of SEQ ID NO: 4, SEQ ID NO: 5, or SEQ ID NO: 11, and wherein the heterologous nucleotide sequence expression is enriched in neurons of the forebrain.

48. The method of claim 38, wherein the vector comprises the nucleotide sequence of SEQ ID NO: 6, and wherein the heterologous nucleotide sequence expression is enriched in neurons of the cerebellum and hippocampus.

49. The method of any one of claims 38-48, wherein the vector, the viral particle, the population, the pharmaceutical composition, or the nanoparticle is administered to the subject intravenously.

50. The method of any one of claims 38-48, wherein the vector, the viral particle, the population, the pharmaceutical composition, or the nanoparticle is administered to the subject via injection into the CNS.
